(19) United States
(12) Patent Application Publication
(10) Pub. No.: US 2005/0272030 A1
(43) Pub. Date: Dec. 8, 2005

(54) Nucleic acid constructs for gene expression

(75) Inventor: Ralph Patrick Braun, Madison, WI (US)

Correspondence Address: FOLEY AND LARDNER;SUITE 500, 3000 K STREET NW, WASHINGTON, DC, 20007, US

(73) Assignee: Powder Ject Research Limited, The Oxford Science Park (GB)

(21) Appl. No.: 10/529,011

(22) PCT Filed: Sep. 29, 2003
(86) PCT No.: PCT/GB03/04218

§ 371 Date: Aug. 2, 2005

Related U.S. Application Data
(60) Provisional application No. 60/414,089, filed on Sep. 27, 2002.

(51) Int. Cl. C12Q 1/70; C12Q 1/68; C07H 21/04

(52) U.S. Cl. 435/5; 435/6; 536/23.72

(57) ABSTRACT

Nucleic acid constructs comprising viral genomic nucleic acid which comprises at least two endogenous gene expression regulatory units which each comprise an endogenous promoter where the endogenous promoters of the units are active at the same phase in the viral life cycle of the virus the viral genomic nucleic acid is derived from are provided. The endogenous gene expression regulatory units are used to express particular chosen heterologous coding sequences and in particular to express heterologous antigens. The constructs can be used in a vaccine to generate an immune response against the heterologous antigens and in particular may be used in a DNA vaccine. Methods for generating the constructs and means for their administration are also provided.

### TECHNICAL FIELD

The present invention relates to the fields of molecular biology and immunology and generally to methods for gene expression. More particularly, the invention pertains to nucleic acid constructs for the expression of polypeptides and their use in eliciting an immune response in a subject by immunization and in particular by nucleic acid immunization.

### BACKGROUND

Conventional vaccination strategies generally involve the administration of either “live” or “dead” vaccines (Ertl et al., (1996) J. Immunol. 156:3579-3582). The so-called live vaccines include attenuated microbes and recombinant molecules based on a living vector. The dead vaccines include those based on killed whole pathogens and subunit vaccines, e.g., soluble pathogen subunits or protein subunits.

Live vaccines are generally successful in providing an effective immune response in immunized subjects; however, such vaccines can be dangerous in immunocompromised or pregnant subjects, can revert to pathogenic organisms, or can be contaminated with other pathogens (Hassett et al., (1996) Trends in Microbiol. 8:307-312). Dead vaccines, such as subunit vaccines, avoid the safety problems associated with live vaccines. As subunit vaccines do not comprise the whole pathogen, they also typically avoid the problem of immunomodulatory viral proteins which may be expressed from attenuated viral vaccines and which can reduce the effectiveness of vaccination. However, dead vaccines, and in particular subunit vaccines, often fail to provide an appropriate and/or effective immune response in immunized subjects.

One possible way of increasing the efficacy of subunit vaccines is for the vaccine to include multiple subunits. Immunization with multiple antigens is desirable, because it typically induces a broader immune response that may give better protection than immunization with a single antigen. Multiple subunit vaccines may also help reduce the need to identify a particular single antigen capable of giving a protective response. This may be particularly important for the induction of cellular immune responses in outbred populations where individuals may vary greatly in their response to a single gene product and hence there is no one antigen is capable of giving rise to a protective immune response in the population as a whole.

Vaccines may be introduced into the subject to be immunized via a number of routes. More recently, direct injection of plasmid DNA by intramuscular (Wolff et al. (1990) Science 247:1465:1468) or intradermal injection with a needle and syringe (Raz et al. (1994) PNAS USA 91:9519-9523) has been described. Thus a construct encoding the antigen, rather than the antigen itself, was introduced into the subject. Such vaccines which comprise a nucleic acid construct encoding the antigen are referred to as DNA vaccines.

Another approach for the delivery of DNA vaccines is referred to as ballistic or particle-mediated DNA delivery and employs a needless particle delivery device to administer DNA-coated microscopic gold beads directly into the cells of the epidermis (Yang et al. (1990) PNAS USA 87:9568-9572). Thus, a number of delivery techniques can be used to deliver nucleic acids for immunizations, including particle-mediated techniques which deliver nucleic acid-coated microparticles into target tissue (see, e.g., co-owned U.S. Pat. No. 5,865,796, issued Feb. 2, 1999).

The level of effective protection achieved with DNA-vaccines is similar to that elicited by traditional protein subunit vaccines and killed or attenuated viral vaccines; although it is traditionally less than that observed in convalescent animals following recovery from a natural infection (Manickan et al. (1997) Critical Review Immunol. 17:139-154). Particle-mediated nucleic acid immunization techniques have been shown to elicit both humoral and cytotoxic T lymphocyte immune responses following epidermal delivery of nanogram quantities of DNA (Pertmer et al., (1995) Vaccine 13:1427-1430). Such particle-mediated delivery techniques have been compared to other types of nucleic acid inoculation, and found markedly superior. Fynan et al. (1995) Int. J. Immunopharmacology 17:79-83, Pynan et al. (1993) Proc. Natl. Acad Sci. USA 90:11478-11482, and Raz et al. (1994) Proc. Natl. Acad. Sci. USA4 91:9519-9523.

### SUMMARY OF THE INVENTION

The present invention is based on the fact that groups of genes in viral genomes have co-evolved in such a way that interference between expression of the genes is minimized, whilst the stability of the region containing the genes is maximized. This co-ordinated evolution of viral genes can be used in the generation of expression constructs for the co-expression of heterologous coding sequences. Thus a region of genomic nucleic acid comprising two or more such viral genes is taken and the natural coding sequences of the viral genes are replaced with the heterologous coding sequences to be expressed. The constructs will therefore benefit from the compatibility of the viral promoters and other regulatory elements used and hence display increased stability and minimal interference. Further modification to the genomic nucleic acid maybe then introduced to optimize the construct.

The present invention therefore provides constructs from which multiple heterologous coding sequences can be expressed. The use of a single construct to express several heterologous polypeptides, rather than expressing each from a separate construct, decreases the complexity of manufacture and quality control. It is also likely to decrease the difficulty of obtaining regulatory approval. The increase in stability of the construct and the decrease in interference due to the use of the endogenous gene expression regulatory units of the viral genomic nucleic acid contrasts with constructs assembled by inserting multiple genes with their own promoters or other promoters commonly used to achieve high level expression into a vector as such constructs often display instability and interference between the promoters.

Accordingly, the present invention provides a nucleic acid construct comprising viral genomic nucleic acid, said viral genomic nucleic acid comprising at least two endogenous gene expression regulatory units which each comprise an endogenous promoter where the endogenous promoters of the units are active at the same phase in the viral life cycle of the virus the viral genomic nucleic acid is derived from, where:

(a) at least two of the endogenous gene expression regulatory units comprising promoters active at the same phase are each operably linked to a separate heterologous coding sequence inserted into the viral genomic nucleic acid; and

(b) the viral genomic nucleic acid is from 1 to 50 kb in length excluding the heterologous sequences inserted into it.

The invention also provides a method of generating a nucleic acid construct for direct administration to a subject to elicit an immune response in the subject, the method comprising:

(a) inserting viral genomic nucleic acid into a vector backbone, said viral genomic nucleic acid comprising at least two endogenous gene expression regulatory units which each comprise an endogenous promoter where the endogenous promoters of the units are active at the same phase in the viral cycle of the virus the viral genomic nucleic acid is derived from; and

(b) either prior to, at the same time, or subsequent to inserting the viral genomic nucleic acid into the vector backbone, operably linking the endogenous promoters of at least two of the endogenous gene expression regulatory units in the viral genomic nucleic acid to heterologous coding sequences

wherein the viral genomic nucleic acid is from 1 to 50 kb in length excluding the heterologous sequences inserted into it.

The invention further provides coated particles, suitable for delivery from a particle-mediated delivery device, which particles comprise carrier particles coated with a nucleic acid construct wherein the construct comprises viral genomic nucleic acid, said viral genomic nucleic acid comprising at least two endogenous gene expression regulatory units which each comprise an endogenous promoter where the endogenous promoters of the units are active at the same phase in the viral cycle of the virus the viral genomic nucleic acid is derived from, where:

(a) at least two of the endogenous gene expression regulatory units comprising promoters are each operably linked to a heterologous coding sequence inserted into the viral genomic nucleic acid; and

(b) the viral genomic nucleic acid is from 1 to 50 kb in length excluding the heterologous sequences inserted into it.

The invention further provides:

a dosage receptacle for a particle mediated delivery device comprising coated particles of the invention; and

a particle mediated delivery device loaded with coated particles of the invention.

The invention also provides in another embodiment a method of obtaining expression in a mammalian cell of a polypeptide of interest, which method comprises transferring into said cells a nucleic acid construct comprising viral genomic nucleic acid, said viral genomic nucleic acid comprising at least two endogenous gene expression regulatory units which each comprise an endogenous promoter where the endogenous promoters of the units are active at the same phase in the viral cycle of the virus the viral genomic nucleic acid is derived from, where:

at least two of the endogenous gene expression regulatory units comprising promoters are each operably linked to a heterologous coding sequence inserted into the viral genomic nucleic acid; and

the viral genomic nucleic acid is from 1 to 50 kb in length excluding the heterologous sequences inserted into it.

In another embodiment the invention provides a method of nucleic acid immunisation comprising administering to a subject an effective amount of coated particles, which particles are suitable for delivery from a particle-mediated delivery device, the particles comprising carrier particles coated with a nucleic acid construct, wherein the construct comprises viral genomic nucleic acid, said viral genomic nucleic acid comprising at least two endogenous gene expression regulatory units which each comprise an endogenous promoter where the endogenous promoters of the units are active at the same phase in the viral cycle of the virus the viral genomic nucleic acid is derived from, where:

at least two of the endogenous gene expression regulatory units comprising promoters are each operably linked to a heterologous coding sequence inserted into the viral genomic nucleic acid; and

the viral genomic nucleic acid is from 1 to 50 kb in length excluding the heterologous sequences inserted into it.

The invention also provides method of generating a nucleic acid construct for direct administration to a subject to elicit an immune response in the subject, the method comprising:

(a) inserting viral genomic nucleic acid into a vector backbone, said viral genomic nucleic acid comprising at least two endogenous gene expression regulatory units which each comprise an endogenous promoter where the endogenous promoters of the units are active at the same phase in the viral cycle of the virus the viral genomic nucleic acid is derived from; and

(b) either prior to, at the same time, or after inserting the viral genomic nucleic acid into the vector backbone, deleting from the viral genomic nucleic acid some or all of the viral sequences, apart from the at least two endogenous gene expression regulatory units, which are present in the region of the viral genome corresponding to that between the 5′ and 3′ ends of the viral genomic nucleic acid of the construct

where the length of the viral genomic nucleic acid inserted into the vector backbone being from 1 to 50 kb.

The present invention also provides a method of obtaining expression in a mammalian cell of a polypeptide of interest, which method comprises transferring into said cells a nucleic acid construct generated by a method of the invention.

The present invention also provides a method of nucleic acid immunisation comprising administering to a subject an effective amount of coated particles, which particles are suitable for delivery from a particle-mediated delivery device, the particles comprising carrier particles coated with a nucleic acid construct generated by a method of the invention.

These and other objects, aspects, embodiments and advantages of the present invention will readily occur to those of ordinary skill in the art in view of the disclosure herein.

### BRIEF DESCRIPTION OF THE DRAWINGS

FIG. 1 provides plasmid maps of cosmid 23 and construct OP23-6 and the various intermediate constructs between the two.

FIG. 2 provides plasmid maps for the constructs OPhsv1-1 and OPhsv1-6 and the various intermediate constructs between the two.

### DETAILED DESCRIPTION OF THE INVENTION

Before describing the present invention in detail, it is to be understood that this invention is not limited to particularly exemplified molecules or process parameters as such may, of course, vary. Thus, for example, the invention is not limited to particular antigens or to antigen encoding nucleotide sequences.

It is also to be understood that the terminology used herein is for the purpose of describing particular embodiments of the invention only, and is not intended to be limiting. In addition, the practice of the present invention will employ, unless otherwise indicated, conventional methods of virology, microbiology, molecular biology, recombinant DNA techniques and immunology all of which are within the ordinary skill of the art. Such techniques are explained fully in the literature. See, e.g., Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.). It is also to be understood that different applications of the disclosed methods may be tailored to the specific needs in the art.

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise.

### DEFINITIONS

Unless defined otherwise, all technical and scientific terms used herein have the same meaning as commonly understood by one of ordinary skill in the art to which the invention pertains. The following terms are intended to be defined as indicated below. Although a number of methods and materials similar, or equivalent to, those described herein can be used in the practice of the present invention, the preferred materials and methods are those described herein.

The term “vaccine composition” intends any pharmaceutical composition containing an antigen (e.g., polynucleotide encoding an antigen), which composition can be used to prevent or treat a disease or condition in a subject. The term thus encompasses both subunit vaccines, i.e., vaccine compositions containing antigens which are separate and discrete from a whole organism with which the antigen is associated in nature, as well as compositions containing whole killed, attenuated or inactivated bacteria, viruses, parasites or other microbes.

The term “nucleic acid immunization” is used herein to refer to the introduction of a nucleic acid molecule encoding one or more selected antigens into a host cell for the in vivo expression of the antigen or antigens. The nucleic acid molecule can be introduced directly into the recipient subject, such as by standard intramuscular or intradermal injection; transdermal particle delivery; inhalation; topically, or by oral, intranasal or mucosal modes of administration. The molecule, alternatively, can be introduced ex vivo into cells which have been removed from a subject. In this latter case, cells containing the nucleic acid molecule of interest are reintroduced into the subject such that an immune response can be mounted against the antigen encoded by the nucleic acid molecule. The nucleic acid molecules used in such immunizations are generally referred to herein as “nucleic acid constructs.”

The term “transdermal” delivery intends intradermal (e.g., into the dermis or epidermis), transdermal (e.g., “percutaneous”) and transmucosal administration, i.e., delivery by passage of an agent into or through skin or mucosal tissue (see, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Bemer (eds.), CRC Press, (1987)). Thus, the term encompasses delivery from a particle delivery device (e.g., needleless syringe) as described in U.S. Pat. No. 5,630,796, as well as particle-mediated delivery as described in U.S. Pat. No. 5,865,796.

By “core carrier” is meant a carrier particle on which a nucleic acid (e.g., DNA) is coated in order to impart a defined particle size as well as a sufficiently high density to achieve the momentum required for cell membrane penetration, such that the nucleic acid can be delivered using particle-mediated delivery techniques, for example those described in U.S. Pat. No.5,100,792. Core carriers typically include materials such as tungsten, gold, platinum, ferrite, polystyrene and latex. See e.g., Particle Bombardment Technology for Gene Transfer, (1994) Yang, N. ed., Oxford University Press, New York, N.Y. pages 10-11.

By “needleless syringe,” is meant an instrument which delivers a particulate composition transdermally, without a conventional needle that pierces the skin. Needleless syringe for use with the present invention are discussed herein.

An “antigen” refers to any agent, generally a macromolecule, which can elicit an immunological response in an individual. The term may be used to refer to an individual macromolecule or to a homogeneous or heterogeneous population of antigenic macromolecules. As used herein, “antigen” is generally used to refer to a protein molecule or portion thereof which contains one or more epitopes. For the purposes of the present invention, antigens can be obtained or derived from any appropriate source. Furthermore, for the purposes of the present invention, an “antigen” includes a protein having modifications, such as deletions, additions and substitutions (generally conservative in nature) to the native sequence, so long as the protein maintains sufficient immunogenicity. These modifications may be deliberate, for example through site-directed mutagenesis, or may be accidental, such as through mutations of hosts which produce the antigens. The immunological response elicited by the antigen may be a cellular antigen-specific immune response, or a humoral antibody response or both. The antigen may, for example, be derived from any known virus, bacterium, parasite, plant, protozoan or fungus. The term “antigen” also includes tumor antigens. The term also includes autoantigens and also antigens from allergens. Similarly, an oligonucleotide or polynucleotide which expresses an antigen, such as in DNA immunization applications, is also included in the definition of antigen. Synthetic antigens are also included, for example, polyepitopes, flanking epitopes, and other recombinant or synthetically derived antigens (Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998).

An “immune response” against an antigen of interest is the development in an individual of a humoral and/or cellular immune response to that antigen. For purposes of the present invention, a “humoral immune response” refers to an immune response mediated by antibody molecules, while a “cellular immune response” is one mediated by T-lymphocytes and/or other white blood cells.

The term “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein.

The term “pathogen” is used in a broad sense to refer to the source of any molecule that elicits an immune response. Thus, pathogens include, but are not limited to, virulent or attenuated viruses, bacteria, fungi, protozoa, parasites, cancer cells and the like. Typically, the immune response is elicited by one or more peptides produced by these pathogens. As described in detail below, nucleic acid encoding the antigenic peptides from these and other pathogens is used to generate an immune response that mimics the response to natural infection. It will also be apparent in view of the teachings herein, that the methods include the use of nucleic acids encoding antigens obtained from more than one pathogen.

The terms “nucleic acid molecule” and “polynucleotide” are used interchangeably to refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, an open reading frame, messenger RNA (mRNA), transfer RNA, ribosomal RNA, ribozymes, cDNA, recombinant polynucleotides, branched polynucleotides, plasmids, cosmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (I) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

A “construct” is any moiety capable of transferring nucleic acids sequences to target cells (e.g., non-viral vectors, particulate carriers, liposomes and viral vectors). A “plasmid” construct is an extrachromosomal genetic element which is capable of self-replication in a host cell. A “cosmid” is a special type of plasmid construct that uses the cos sequences of bacteriophage lambda (λ). The term “cos ends” or “cos sites” refers to the single stranded 12 base pair complementary extensions of λ DNA Cosmids can carry large inserts, for example up to around 50 kb in size, whilst typical plasmids can carry inserts under about 10 kb in size. Because of their capacity to carry large fragments, cosmids are useful for the construction of genomic libraries and also for situations where large inserts are needed. Typically, “vector,” “construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

A “genomic library” is a collection of recombinant nucleic acid molecules which together represent the entire, or almost entire, genome of an organism. In cases where the library has almost, but not quite, all of the genome, it may, for example, comprise more than 95%, 98%, 99% or even 99.9% of the sequences in the genome.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, procatyotic or eucaryotic mRNA, genomic DNA sequences from viral or procaryotic DNA, and even synthetic DNA sequences. A transcription termination sequence may be located 3′ to the coding sequence. Transcription and translation of coding sequences are typically regulated by “control elements,” including, but not limited to, transcription promoters, transcription enhancer elements, Shine and Delagarno sequences, transcription termination signals, polyadenylation sequences (located 3′ to the translation stop codon), sequences for optimization of initiation of translation (located 5′ to the coding sequence), and translation termination sequences. A “gene expression regulatory unit” refers to a nucleotide sequence comprising, as a minimum, a promoter. The unit may additionally comprise other sequences which are needed for, or influence, expression of the coding sequences operably linked to the promoter. The elements of the unit do not have to be contiguous and may be separated by intervening sequences. The elements fo the unit may influence expression of the coding sequences at the level of transcription, RNA stability, RNA processing and/or translation. Typically, the unit does not include the coding sequences to which it is operably linked. In some cases a gene expression regulatory unit may comprise, or consist essentially of, a naturally occurring gene apart from the coding sequences of the gene.

A “promoter” is a nucleotide sequence which directs transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

An “endogenous gene expression regulatory unit” refers to a gene expression regulatory unit which is derived from the same organism as some, or all, of the nucleic acid sequences it is present in Thus an endogenous gene expression regulatory unit will typically have sequences either upstream or downstream of it, or both, which are derived from the same organism as the gene expression regulatory unit itself and which preferably correspond to some, or all, of the sequences which flank the gene expression regulatory unit in the genome of that organism. Typically, some, or all, of the flanking sequences derived from the same organism as the gene expression regulatory unit may have the same position relative to the gene expression regulatory unit that they do in the genome of the organism and may be immediately upstream and/or downstream of the gene expression regulatory unit. Thus, in the case of the nucleic acid constructs of the invention, the endogenous gene expression regulatory unit will typically originate from, and be part of, the viral genomic nucleic acid sequences present in the construct.

A “heterologous coding sequence” is a coding sequence operably linked to a gene expression regulatory unit, and in particular a promoter, with which it is not naturally associated. Typically, the two will have been operably linked via recombinant DNA techniques. The heterologous coding sequence may originate from the same organism as the endogenous gene expression regulatory unit it is operably linked to or, alternatively, it may be from a different organism to the gene expression regulatory unit it is linked to. The heterologous coding sequence may, for example, be any of the coding sequences mentioned herein and in particular may encode a heterologous antigen.

An “isolated polynucleotide” molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith. A sequence is “derived or obtained from” a molecule if it has the same or substantially the same basepair sequence as a region of the source molecule, its cDNA, complements thereof, or if it displays sequence identity as described below.

“Operably linked” refers to an arrangement of elements wherein the components so described are configured so as to perform their usual function. Thus, a given gene expression regulatory unit, and in particular a promoter, that is operably linked to a coding sequence (e.g., encoding an antigen of interest) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Recombinant“as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. Recombinant includes both DNA and RNA molecules falling within this definition. The term “recombinant” as used with respect to a protein or polypeptide means a polypeptide produced by expression of a recombinant polynucleotide.

Homologues of polynucleotides are referred to herein. Typically a polynucleotide which is homologous to another polynucleotide is at least 70% homologous to the polynucleotide, preferably at least 80 or 90% and more preferably at least 95%, 97% or 99% homologous thereto. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60 or 100 or more contiguous nucleotides.

Methods of measuring polynucleotide homology or identity as well as polypeptide homology or identity are known in the art. For example the UWGCG Package provides the BESTFIT program which can be used to calculate homology (e.g. used on its default settings) (Devereux et al (1984) Nucleic Acids Research 12, p387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifyig short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (13) of 50, expectation () of 10, M=5, N=4, and a comparison of both strands.

The BLAST algorithm performs a statistical analysis of the similarity between two sequences; see e.g., Karlin and Altschul (1993) Proc. Natl, Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologues typically hybridize with the relevant polynucleotide at a level significantly above background. The signal level generated by the interaction between the homologue and the polynucleotide is typically at least 10 fold, preferably at least 100 fold, as intense as “background hybridisation”. The intensity of interaction may be measured, for example, by radiolabelling the probe, e.g. with ³²P. Selective hybridisation is typically achieved using conditions of medium to high stringency, (for example, 0.03M sodium chloride and 0.003M sodium citrate at from about 50° C. to about 60° C..

Stringent hybridization conditions can include 50% formamide, 5× Denhardt's Solution, 5×SSC, 0.1% SDS and 100 μpg/ml denatured salmon sperm DNA and the washing conditions can include 2×SSC, 0.1% SDS at 37° C. followed by 1×SSC, 0.1% SDS at 68° C.. Defining appropriate hybridization conditions is within the skill of the art. See, e.g., Sambrook et al., supra.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue. Where a polynucleotide encodes a polypeptide, substitutions preferably create “conservative” changes in the amino acid encoded. These are defined according to the following table. Amino acids in the same block in the second column and preferably in the same line in the third column may be substituted for each other in conservative changes.

| | | |
|---|---|---|
| ALIPHATIC | Non-Polar | G A P |
| | | I L V |
| | Polar-uncharged | C S T M |
| | | N Q |
| | Polar-charged | D E |
| | | K R |
| AROMATIC | | H F W Y |

As used herein the term “adjuvant” refers to any material that enhances the action of a drug, antigen, polynucleotide, vector or the like. It is intended, although not always explicitly stated, that molecules having similar biological activity as wild-type or purified peptide adjuvants (e.g., recombinantly produced or muteins thereof) and nucleic acid encoding these molecules are intended to be used within the spirit and scope of the invention.

As used herein, the term “treatment” includes any of following: the prevention of infection or reinfection; the reduction or elimination of symptoms; the reduction or complete elimination of a pathogen; and the reduction, prevention, amelioration or elimination of a disease or disorder. Treatment may be effected prophylactically (e.g. prior to infection) or therapeutically (e.g. following infection).

The terms “individual” and “subject” are used interchangeably herein to refer to any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

### GENERAL OVERVIEW OF THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

The present invention is concerned with nucleic acid constructs which allow expression of multiple antigens from the same construct and the use of these constructs for nucleic acid immunization. The invention also provides methods for the construction of such constructs. The constructs comprise viral genomic nucleic acid and utilize two or more of the endogenous gene expression regulatory unit present in the viral genomic nucleic acid to express the desired heterologous polypeptides. The heterologous coding sequences to be expressed are inserted into the constructs so that they are operably linked to the chosen endogenous gene expression regulatory units and in particular to the endogenous promoters of these units. The constructs allow for efficient expression of the heterologous coding sequences, and in particular antigen-encoding genes, in host cells.

The nucleic acid constructs of the present invention typically comprise, or in some embodiments consist essentially of, viral genomic nucleic acid, said viral genomic nucleic acid comprising at least two endogenous gene expression regulatory units which each comprise an endogenous promoter where the endogenous promoters of the units are active at the same phase in the viral life cycle of the virus that the viral genomic nucleic acid is derived from, where:

(a) at least two of the endogenous gene expression regulatory units comprising promoters active at the same phase are each operably linked to a separate heterologous coding sequence inserted into the viral genomic nucleic acid; and

(b) the viral genomic nucleic acid is from 1 to 50 kb in length excluding the heterologous sequences inserted into it.

Advantages of the present invention include, but are not limited to, (i) high construct stability and low interference between genes; (ii) providing an array of antigens (e.g., epitopes) rather than a single antigen so that the construct more closely mimics that of a natural infection; (iii) achieving co-delivery of the antigens into the same cell to achieve coordinated expression of multiple antigens, (iv) eliciting an immune response similar to that elicited by natural infection due to the expression of multiple antigens; (v) eliciting an immune response that is more protective than that elicited by natural infection, e.g., because the antigens chosen do not include immunodominant antigens or polypeptides that inhibit immune responses which may be present in whole viral vaccines; and (vi) triggering the antigen processing and presentation pathways that are normally involved in the clearance of intracellular infections.

Endogenous Gene Expression Regulatory Units

The viral genomic nucleic acid in a construction of the invention comprises at least two endogenous gene expression regulatory units. Each endogenous gene expression regulatory unit will comprise an endogenous promoter. Typically, however, the unit will comprise other nucleotide sequences. In particular, the until will comprise nucleotide sequences necessary for, or which influence, transcription and/or translation of the coding sequences with which the endogenous gene expression regulatory unit is operably linked. In many embodiments one or more of the units will comprise, or consist essentially of, an endogenous gene in which the coding sequences naturally associated with the gene have been replaced with a heterologous coding sequence.

Each endogenous gene expression regulatory unit may comprise elements essential for, or which influence, transcription from the coding sequences operably linked to the unit. These may include an enhancer sequence or other sequence which modulates expression of the operably linked coding sequences. Sequences which modulate the conformation and/or accessibility of the coding sequences and/or other sequences may be present in the unit. In a preferred embodiment the endogenous transcriptional termination sequence may also be present.

As a minimum the endogenous gene expression regulator unit will comprise an endogenous promoter. Typically this will be the entire endogenous promoter i.e. the endogenous sequences necessary to achieve normal expression of the coding sequences which the endogenous promoter is naturally operably linked and/or the same specificity and level of expression of the heterologous coding sequences to which the unit will be operably linked in the construct of the invention. In some embodiments the promoter may have had some sequence modifications made to it. For example, base substitutions, insertions or deletions may have been introduced such as, for example, none, two, five, ten or more base substitutions, insertions and/or deletions. In other embodiments larger modifications may have been introduced. For example, deletions of from two to five, from five to ten, from ten to twenty or more bases may have been introduced. In some cases the promoter may have been truncated to the minimal sequences necessary to achieve expression of the coding sequences it is operably linked to although typically this will not be the case. In some embodiments the endogenous sequences between the promoter and transcriptional start site will be retained.

The endogenous gene expression regulatory units may also comprise sequences which are involved in, or influence, translation. The transcribed, non translated sequences associated with the endogenous gene from which the unit is derived may be present in the unit. For example some, or all, of the as translated 5′ and/or 3′ regions of the unit may be retained. In particular regions which influence transcript processing and/or stability may be retained. The shine Dalgarno, start codon and/or stop codon may also be retained. Sequences which influence transcript conformation may be retained in the unit and in particular where these influence the level of expression of the coding sequences operably linked to the unit.

The endogenous gene expression regulatory unit does not have to comprise all of the non coding sequences from the endogenous gene it is derived from, although it may do. It may comprise the endogenous promoter and, optionally, any other region from the endogenous gene apart from the coding sequences. It may comprise the endogenous promoter in combination with one or more of the endogenous gene elements mentioned herein. The endogenous gene expression regulatory unit may comprise sequences which are spatially separated in the endogenous gene.

Some of the sequences in the non coding regions of the endogenous gene may be absent from the unit, such as sequences which do not play a role in, or influence, transcription and/or translation. In some cases regulatory elements naturally associated with the heterologous coding sequence may be used rather than their counterparts from the endogenous gene which the gene expression regulatory unit is derived. For example, the 5′ or 3′ untranslated regions of the transcript may be those naturally associated with the heterologous coding sequences. Introns may originate from the same source as the heterologous coding sequences. In some cases regulatory regions from heterologous sources different to the origin of the heterologous coding sequences may be employed.

The endogenous gene expression regulatory units may originate from any suitable viral gene and in particular form any viral gene mentioned herein.

The viral genomic nucleic acid in a construct of the invention comprises at least two endogenous gene expression regulatory units and may, for example, comprise two, three, four, five or more endogenous gene expression regulatory units. At least two of the endogenous gene expression regulatory units comprise an endogenous promoter expressed at the same phase in the viral life cycle of the virus that the viral genomic nucleic acid originates from and preferably three, four, five or more of the endogenous gene expression regulatory units comprise such promoters expressed at the same phase in the viral life cycle. In some embodiments, all of the endogenous promoters of the units may be expressed at the same phase. At least two of the endogenous promoters expressed at the same phase in the viral life cycle will be individually operably linked to a heterologous coding sequence and preferably three, four, five or more of the promoters may be so operably linked to heterologous coding sequences. In some embodiments, all of the endogenous promoters of the units may be separately linked to heterologous coding sequences.

The endogenous gene expression regulatory units will typically have the same origins and be part of the viral genomic nucleic acid that they are present in. Thus preferably the viral genomic nucleic acid will be obtained from the viral genome as a single fragment and will then be subsequently modified to introduce the heterologous coding sequences and to make any other modifications so desired. Although this is the preferred route for the generation of constructs of the invention other routes which achieve the same end result, such as obtaining the viral genomic nucleic acid as several fragments and assembling them stepwise with additional sequence being inserted at the same time, or later on, into an appropriate vector, are also encompassed by the invention.

In many embodiments the endogenous gene expression regulatory units and in particular the endogenous promoter of the unit may have the same sequences upstream and/or downstream of it that it did in the viral genome. In particular, the endogenous gene expression regulatory units may have some or all of the same upstream sequences that it did in the viral genome, within the limits of the viral genomic nucleic acid in the construct. In some embodiments some, or all, of the sequences downstream of the heterologous coding sequence that the unit, and in particular the endogenous promoter, is operably linked to may be equivalent to those downstream of the coding sequence which the unit, and in particular the endogenous promoter, is naturally associated with. The endogenous downstream elements present in the unit may include elements which are included in the transcript from the endogenous promoter such as, for example, those involved with determining the stability of the transcript. The endogenous downstream sequences present may comprise a transcriptional termination element and/or a polyadenylation signal. Sequences upstream and/or downstream of the heterologous coding sequences or in intronic sequences within the coding sequences may include endogenous enhancer elements. Upstream sequences present may include the endogenous Shine and Dalgarno sequence. In some embodiments the whole of the endogenous gene, that the endogenous endogenous gene expression regulatory unit is derived from, is retained in the construct apart from the coding sequences. In addition, any sequences which affect expression from the endogenous promoter of the unit, such as an enhancer, may also be retained.

In some embodiments the sequences for more than 100 base pairs, preferably more than 500 bp, more preferably more than 1 kb and even more preferably more than 2 kb upstream of the unit, and in particular of the promoter, and/or downstream of the heterologous coding sequence may be homologous or identical to those upstream and/or downstream of the unit, and in particular of the endogenous promoter and its coding sequence in the viral genome. The region of upstream and/or downstream sequence identity to those in the viral genome may extend to the next endogenous gene expression regulatory unit operably linked to heterologous coding sequences and/or to the next heterologous coding sequences. In some embodiments there may be deletions present in the viral genomic nucleic acid in the construct upstream and/or downstream of the endogenous gene expression regulatory unit and the heterologous coding sequence which means that the sequences upstream and/or downstream which are equivalent to some of those found upstream and/or downstream in the viral genome may, in effect, be moved closer to the endogenous gene expression regulatory unit in the construct.

The endogenous promoters, to which the heterologous coding sequences are operably linked, will preferably be expressed in the same phase and typically at a similar, or same, time in the viral cycle of the virus the viral genomic nucleic acid is derived from. Viral life cycles are typically divided into phases, each of which may involve the expression of a particular subset of genes, the genes being classified as to what phase they are expressed at. For example, a viral life cycle may involve immediate early, early and late gene expression or gene expression during a period of latency. Thus in many embodiments the endogenous promoters of the gene expression regulatory units will be those of viral genes from the same or adjacent phases in the viral life cycle and preferably the same phase. Thus they may all be immediate early, early, late or latency associated promoters.

Typically, at some stage in the viral life cycle the endogenous promoters will both/all be being expressed that is there will be an overlap or when the promoters give rise to transcription. Preferably the time at which transcription starts and/or ceases from the endogenous promoters linked to the heterologous coding sequences will occur at a similar or identical time point.

In some cases, he promoters chosen may be both expressed in the same phase of viral gene expression such as, for example, the immediate early phase, but there will not be an actual overlap in hen the promoters are expressed rather all the promoters will be expressed sequentially in the same phase.

In some embodiments of the invention immediate early promoters may be chosen to express the heterologous coding sequences. This may, in particular, be the case where viral proteins are required for expression from promoters from later stages in the viral life cycle. Hence, preferably, the promoters chosen will not require viral proteins for expression.

In other embodiments the promoters may be chosen so as to mimic a particular stage in the vial life cycle. Thus by using immediate early gene promoters it may be possible to mimic the situation where a virus, such as HSV for example, emerges from a period of latency.

Examples of preferred sets of endogenous promoters include:
(i) At least two of the ICPO, 4, 22 and 27 genes of HSV and in particular: ICPO and 4; ICP4 and 22; ICP22 and 27; ICP 0,4 and 22; ICP 4,22 and 27; or ICP 0,4,22 and 27.
(ii) At least two of the HSV tegument promoters and in particular two of UL48, 49 and 50, such as: UL48 and UL49; UL49 and 50; or UL48,49 and 50.
(iii) At least two of: UL83 and UL84; UL122 and UL123; or UL36, 37 and 38 of cytomegalovirus and in particular human cytomegalovirus such as: WL36 and 37; or UL37 and 38.

In many cases viral genes which are expressed at a similar stage in the viral life cycle are adjacent to each other with no intervening genes between them. In many embodiments the endogenous gene expression regulatory units chosen will therefore be those originating from adjacent or closely linked genes in the viral genome from which they are derived. Thus the endogenous gene expression regulatory units chosen to drive expression of the heterologous coding sequences may be those derived from two, three, four or more consecutive genes in the viral genome, although in some embodiments there may be intervening genes, such as one, two or three or more for example, between two of the endogenous gene expression regulatory units chosen.

Some viruses contain repeated sequences within their genome. For example HSV has two sets of such repeats. In many embodiments of the invention the viral genomic nucleic acid present in the construct will not comprise a repeated sequence and in particular will not comprise multiple copies of the same gene, unit or promoter. Preferably, the construct will not comprise inverted repeats in the the viral genomic nucleic acid and in particular will not comprise inverted repeats of genes, promoters and or units or inverted repeats of two homologous promoters, genes or units.

Preferably, the heterologous coding sequences operably linked to the endogenous promoters will not have any of the promoter elements with which they are naturally operably linked. Thus, the promoter responsible for their expression will be the endogenous viral promoter of the endogenous gene expression regulatory unit and not one inserted, for example, with the heterologous coding sequences. However, in some embodiments some or all of the promoter elements naturally linked to the heterologous coding sequences may be introduced upstream of the endogenous promoter or downstream of it, but upstream of the transcriptional start point. In such embodiments, typically the heterologous promoter will be downstream of the endogenous one.

Typically, the heterologous coding sequences will be inserted in place of the coding sequences with which the promoter is naturally associated. Thus the coding sequences naturally linked to the endogenous promoter will typically be deleted and replaced with the heterologous coding sequence. In some embodiments, the first few codons of the natural coding sequences may remain and be fused to the heterologous coding sequences. Any arrangement leading to the expression of the polypeptide encoded by the heterologous sequences may be used. In one embodiment the coding sequences naturally associated with the promoter may remain with the heterologous coding sequences downstream of them with an internal ribosome entry sequence (IRES) to ensure translation.

In some embodiments the constructs may comprise two or more sets of endogenous gene expression regulatory units operably linked to heterologous coding sequences. Each set of gene expression regulatory units operably will comprise at least two promoters expressed at the same phase in the life cycle of the virus that the viral genomic nucleic of the construct is derived from. The different sets of units will give rise to expression at different times. This will allow expression of particular antigens at different times.

Antigens

The heterologous coding sequences present in the constructs of the invention will typically encode antigens. The methods and constructs described herein are useful in eliciting an immune response against a wide variety of cells, tissues and human or animal pathogens. These pathogens comprise one or more antigens. The heterologous polypetides expressed by a construct of the invention may be one or more of any of these antigens. Non-limiting examples of sources for antigens to be expressed by the constructs of the invention include viruses, bacterial cells, fungal cells, parasites and other pathogenic organisms. In many embodiments the antigens will be derived from a disease causing infectious agent.

The antigens encoded by the heterologous coding sequences in the constructs of the invention may originate from the same organism as the viral genomic nucleic acid of the construct or from a different organism. They may all originate from the same organism or two or more, or all, of them may originate from different organisms. The antigens may originate from several closely related organisms. Thus, for example, they may originate from several strains of the same pathogen, the aim being to immunise a subject so that a protective response may be generated against each of the strains that the antigens originate from. They may encode the equivalent antigen from each strain.

Typically, the heterologous coding sequences will encode different antigens, although in some embodiments two or more, or even all, of the heterologous coding sequences may encode the same antigen in order to obtain a higher level of expression of the antigen.

The antigens expressed by the construct may occur in similar locations in the pathogen and/or have similar functions. For example, they may all be expressed on the surface of the pathogen or alternatively may be antigens which are not exposed on the surface of the pathogen such as intracellular antigens. The antigens may all be viral coat proteins, glycoproteins or other proteins expressed on the surface of a virus. In some embodiments, a construct may express both a surface antigen and a non-surface antigen.

In some embodiments the antigen will be part of a fusion protein expressed from the endogenous promoter. Thus the antigenic sequences may be fused to those normally expressed by the endogenous promoter of the gene expression regulatory unit. Alternatively, the endogenous promoter may drive the expression of a fusion protein comprising, or in some embodiments consisting essentially of, several different antigens or epitopes. The fusion protein may comprise any combination of two or more of the antigens discussed herein. In addition to the sequences coding the antigen the heterologous coding sequences inserted may also include sequences to target the antigens to the appropriate site. They may also include cleavage sites for specific proteases to allow release of specific antigens or sequences from the fusion.

In some embodiments of the invention the antigens will mainly be, or all be, those thought to give rise to primarily a cellular or humoral response such that the response is mainly or almost all cellular or humoral. Thus the antigens may be ones not present on the surface of the pathogen, for example they may not be glycoproteins, in an effort to generate a primarily cell mediated response rather than a humoral one. In some embodiments the reverse may be true. In some embodiments the antigens may be chosen to specifically elicit both a cellular and a humoral response.

Suitable viral antigens include, but are not limited to, those obtained or derived from the hepatitis family of viruses, including hepatitis A virus (HAV), hepatitis B virus (HBV), hepatitis C virus (HCV), the delta hepatitis virus (HDV), hepatitis E virus EV) and hepatitis G virus (HGV). See, e.g., WO 89/04669; WO 90/11089; and WO 90/14436. The HCV genome encodes several viral proteins, including E1 and E2. See, e.g., Houghton et al. (1991) Hepatology 14:381-388. Similarly, the coding sequence for the 8-antigen from HDV is known (see, e.g., U.S. Pat. No. 5,378,814).

In like maimer, a wide variety of proteins from the herpesvirus family can be used as antigens in the present invention, including proteins derived from herpes simplex virus (HSV) types 1 and 2, such as HSV-1 and HSV-2 glycoproteins gB, gD and gH; antigens from varicella zoster virus (VZV), Epstein-Barr virus (EBV) and cytomegalovirts (CMV) including CMV gB and gH; and antigens from other human herpesviruses such as HHV6 and HHV7. (See, e.g. Chee et al. (1990) Cytomegalovirmses (J. K. McDougall, ed., Springer-Verlag, pp. 125-169; McGeoch et al. (1988) J. Gen. Virol. 69:1531-1574; U.S. Pat. No. 5,171,568; Baer et al. (1984) Nature 310:207-211; and Davison et al. (1986) J. Gen. Virol. 67:1759-1816.) Human immunodeficiency virus TV) antigens, such as gp 120 molecules for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); and Modrow et al. (1987) J. Virol. 61:570-578) and antigen-encoding sequences derived or obtained from any of these isolates will find use in the present invention. Furthermore, other immunogenic proteins derived or obtained from any of the various HIV isolates may be an antigen expressed by a construct of the invention, including one or more of the various envelope proteins or fragments thereof such as gp160 and gp41, gag antigens such as p24gag and p55gag, as well as proteins derived from the pol, env, tat, vif, rev, nef, vpr, vpu and LTR regions of HIV.

Antigens derived or obtained from other viruses will also find use herein, such as without limitation, antigens from members of the families Picornaviridae (e.g., poliovinises, rhinoviruses, etc.); Caliciviridae; Togaviridae (e.g., rubella virus, dengue virus, etc.); Flaviviridae; Coronaviridae; Reoviridae (e.g., rotavirus, -etc.); Birnaviridae; Rhabodoviridae (e.g., rabies virus, etc.); Orthomyxoviridae (e.g., influenza virus types A, B and C, etc.); Filoviridae; Paramyxoviridae (e.g., mumps virus, measles virus, respiratory syncytial virus, parainfluenza virus, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV_{IIIb}, HIV_{SF2}, HIV_{LAV}, HIV_{LaI}, HIV_{MN}); HIV-1_{CM235}, HIV-1_{US4}; HIV-2, among others; simian immunodeficiency virus (SIV); Papillomavirus, the tick-bourne encephalitis viruses; and the like. See, e.g. Virology, 3rd Edition (W. K. Joklik ed. 1988); Fundamental Virology, 2nd Edition (B. N. Fields and D. M. Knipe, eds. 1991), for a description of these and other viruses.

In some contexts, it may be preferable that the selected antigens are viral antigens obtained or derived from a viral pathogen that typically enters the body via a mucosal surface and is known to cause or is associated with human disease, such as, but not limited to, HIV (AIDS), influenza viruses (Flu), herpes simplex viruses (genital infection, cold sores, STDs), rotavimses (diarrhea), parainfluenza viruses (respiratory infections), poliovirus (poliomyelitis), respiratory syncytial virus (respiratory infections), measles and mumps viruses (measles, mumps), rubella virus (rubella), and rhinoviruses (common cold).

Bacterial and parasitic antigens which may be encoded by the heterologous coding sequences of the constructs of the invention include those obtained or derived from known causative agents responsible for diseases including, but not limited to, Diptheria, Pertussis, Tetanus, Tuberculosis, Bacterial or Fungal Pneumonia, Otitis Media, Gonnorhea, Cholera, Typhoid, Meningitis, Mononucleosis, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borelia, and Trichinosis.

Still further antigens can be obtained or derived from unconventional viruses such as prions including the causative agents of kuru, Creutzfeldt-Jakob disease (CJD), scrapie, transmissible mink encephalopathy and chronic wasting diseases, or from, the prions that are associated with mad cow disease. They may also be, or be derived from the prions responsible for familial fatal insomnia In prion diseases, where there may be a particular conformational form of prion protein associated with the disease as well as a normal conformational form, preferably the antigen expressed by the construct will be such that a response is only raised against the disease associated conformational form of the prion protein and not the normal form of the protein.

Specific pathogens which antigens may be derived from can include M. tuberculosis, Chlamydia, N. gotiorrhoeae, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pneumophila, Yersinia pestis, Streptococcus (types A and B), Pneumococcus, Meningococcus, Hemophilus influenza (type b), Toxoplasma gondic, Complylobacteriosis, Moraxella catarrhalis, Donovanosis, and Actinomycosis; fungal pathogens including Candidiasis and Aspergillosis; parasitic pathogens including Taenia, Flukes, Roundworms, Amebiasis, Giardiasis, Cryptosporidium, Schistosoma, Pneumocystis carinji, Trichomoniasis and Trichinosis. Thus, the present invention can also be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral diarrhea virus (BVDV), Klebsiella pneuinoniae, E. coli, Bordetella pertussis, Bordetella parapertussis and brochiseptica.

In some embodiments one or more, and preferably all, of the antigens expressed by the construct of the invention will be tumor antigens. Preferably these antigens will be specific to tumors and will not be expressed by other cell types or at least other cell types of the subject. Such antigens may be derived from malignant tumors and in particular metastatic tumors. In some cases the antigens will have been specifically isolated from the subject to be treated or matched to the specific tumor antigens expressed by the tumor of the subject.

In other embodiments the antigen may be an autoantigen and in particular an autoantigen implicated in or responsible for an autoimmune disease or disorder. Alternatively the antigen may be, or derived from, an allergen.

Adjuvants

In some embodiments, the present invention may effectively be used with any suitable adjuvant or combination of adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); adjuvants formed from bacterial cell wall components such as adjuvants including lipopolysaccharides (e.g., lipid A or monophosphoryl lipid A (MPL), Inoto et al. (1985) Tet. Lett. 26:1545-1548), trehalose dimycolate (TDM), and cell wall skeleton (CWS); heat shock protein or derivatives thereof; adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, Staphylococcus aureus EDIN, and ADP-ribosylating bacterial toxin mutants such as CRM,₉₇, a non-toxic dliphtheria toxin mutant (see, e.g., Bixler et al. (1989) Adv. Exp. Med. Biol. 251:175; and Constantino et al. (1992) Vaccine); saponin adjuvants such as Quil A (U.S. Pat. No. 5,057,540), or particles generated from saponins such as ISCOMs (immunostimulating complexes); chemolines and cytokines, such as interleukins (e.g., IL-1, IL-2, IL-4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1-α and MIP-2, etc; muramyl peptides such as N-acetyl-muramyl-L-threonyl-D-isoglutamine (thr-MDP), N-acetyl-normuramyl-^{L}-alanyl-^{D}-isoglutanine (nor-MDP), N-acetylmuramyl-^{L}-alanyl-^{D}-isoglutaminyl-^{L}-alanine-2-(1′-2′-dipalirtoyl-sn-glycero-3 huydroxyphosphoryloxy)-ethylamine (MTP-PE) etc.; adjuvants derived from the CpG family of molecules, CpG dinucleotides and synthetic oligonucleotides which comprise CpG motifs (see, e.g., Krieg et al. Nature (1995) 374:546, Medzbitov et al. (1997) Curr. Opin. Immunol 9:4-9, and Davis et al. J. Immunol. (1998) 160:870-876) such as TCC ATG ACG TTC CTG ATG CT (SEQ ID NO: 1) and ATC GAC TCT CGA GCG TTC TC (SEQ ID NO: 2); and synthetic adjuvants such as PCPP (Poly[di(carboxylatophenoxy)phosphazene) (Payne et al. Vaccines (1998) 16:92-98). Such adjuvants are commercially available from a number of distributors such as Accurate Chemicals c; Ribi Inmunechemicals, Hamilton, Mont.; GIBCO; Sigma, St. Louis, Mo.

A preferred adjuvant for use in the present invention is imiquimod. Imiquimod is 1-(2-methyl-propyl)-1H-imidazo[4,5-c]quinolin-4-amine. It has a molecular formula of C₁₄H₁₆N₄ and a molecular weight of 240.3. Imiquimod has the following structure:

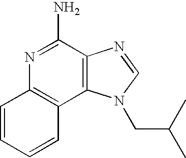

Another preferred adjuvant is resiquimod:

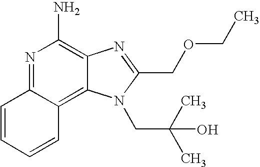

Resiquimod is 4-amino-2-ethoxymethyl-alpha, alpha-dimethyl-1H-imidazo [4,5-c] quinoline-1-ethanol. (R-848; S-28463). Suitable derivatives of imiquimod and resiquimod may be used.

The adjuvant may delivered individually or delivered in a combination of two or more adjuvants. In this regard, combined adjuvants may have an additive or a synergistic effect in promoting an immune response. A synergistic effect is one where the result achieved by combining two or more adjuvants is greater than one would expect than by merely adding the result achieved with each adjuvant when administered individually.

The adjuvant may be expressed from a nucleic acid construct administered to the subject. The adjuvant maybe encoded by the construct fo the invention or by a separate construct. The constructs fo the invention may therefore include a region encoding an adjuvant, operably linked to regulatory elements allowing expression of the adjuvant in the subject.

In embodiments where the adjuvant is encoded by a nucleic acid any suitable gene expression regulatory unit may be employed to express the adjuvant. Promoters which give rise to high level constitutive expression of the adjuvant may be employed. Alternatively, similar or identical gene expression regulatory units to those used to express the antigen maybe employed.

In embodiments where the adjuvant is encoded on a separate construct from the nucleic acid construct of the invention, the construct encoding the adjuvant will preferably be administered with, at the same time, as or in sequence with the construct of the invention. Typically the two will be administered as a single composition. For example the two constructs may be coated onto the same particles or alternatively on separate particles and then mixed. Compositions comprising such particles ro mixtures of particles are provided by the invention.

In embodiments where the adjuvant is encoded by a nucleic acid which is to be administered to the subject, examples of preferred adjuvants include any polypeptide adjuvants mentioned herein and, in particular PT, CT, LT and DT.

Preparation of Viral Genomic Nucleic Acid

Typically, the viral genomic nucleic acid for use in the invention will be obtained from a genomic library of the particular virus chosen. Other methods such as PCR amplification of the chosen region of the viral genome either as several fragments or as a single fragment or obtaining the region from existing clones of a subregion of the viral genome may also be employed.

Viral genomic libraries can be produced by any method known in the art. In many embodiments of the invention the viral genomic nucleic acid in the construct of the invention may be a fragment from a genomic library or may be derived from such a fragment. A variety of sources can be used for the genomic DNA. Genomic DNA may be commercially available, for example, from sources such as Advanced Biotechnologies Inc (ABI) and Clonetech, Inc. Another standard source is genomic DNA directly isolated from the virus chosen.

The viral genomic nucleic acid used in a construct of the invention, and also the construct itself, may be double or single stranded nucleic acid and may be RNA or DNA. In embodiments of the invention where an RNA virus is used the RNA may be first converted to DNA and then manipulated in that form before an RNA construct is then generated from the DNA.

Genomic DNA from the selected source can be isolated by standard procedures, which typically include successive phenol and phenol/chloroform extractions followed by ethanol precipitation. After precipitation, the DNA from a virus of interest can be treated with a restriction endonuclease. The digestion with the restriction endonuclease may be deliberately partial in order to obtain longer fragments. Alternatively, the genomic DNA may be digested to completion. The restriction enzyme employed may be chosen on the basis of the average frequency with which it cuts DNA so that a large portion of the fragments in the resulting digest are within a certain desired size range. DNA fragments of a selected size can be separated by a number of techniques, including agarose or polyacrylamide gel electrophoresis or pulse field gel electrophoresis (Carle et al. (1984) Nuc. Acid Res. 12:5647-5664; Chu et al. (1986) Science 234:1582; Smith et al. (1987) Methods in Enzymology 151:461), to provide an appropriate size starting material for cloning.

The genomic fragments may be blunt ended and cloned into a similarly blunt ended vector or may have particular single stranded overhangs from restriction enzyme cleavage and hence can be cloned into a vector which has been prepared to give compatible overhangs. Restriction cleaved fragments may be blunt ended, if desired, by treating with the large fragment of E. coli DNA polymerase I (Klenow) in the presence of the four deoxynucleotide triphosphates (dNTPs) using standard techniques. The Klenow fragment fills in at 5′ single-stranded overhangs, but digests protruding 3′ single strands, even though the four dNTPs are present. If desired, selective repair can be performed by supplying only one, or several, selected dNTPs within the limitations dictated by the nature of the overhang. After Kienow treatment, the mixture can be extracted with e.g. phenol/chloroform, and ethanol precipitated. Treatment under appropriate conditions with S1 nuclease or BAL-31 results in hydrolysis of any single-stranded portion in restriction fragments also yielding blunt ended fragments.

Once suitable genomic fragments have been prepared they may be cloned into any suitable vector construct or replicon. Examples of suitable vectors are well known in the art. The vector may, for example, be a plasmid or in some embodiments of the invention may be a cosmid. When using cosmid cloning vectors, the genomic nucleic acid fragments cloned into them are typically large, preferably between about 20,000 bp (20 kb) and 50,000 base pairs (50 kb) in size (or any integer there between), preferably between about 25 kb and 50 kb, more preferably between about 30-35 kb and 50 kb, and even more preferably between about 35 kb and about 50 kb. Suitable cosmid vectors are commercially available, for example the SuperCos 1 Cosmid Vector Kit (Stratagene, La Jolla, Calif.). Ligation of the DNA into the cosmid is performed as instructed by the manufacturer or may be empirically determined using methods known in the art in view of the teachings of this specification.

In another preferred embodiment, the viral genomic fragments are cloned into plasmids to generate plasmid libraries. When using plasmid cloning vectors, the fragments are typically between about 5,000 bp (5 kb) and 25,000 base pairs (25 kb) in size (or any integer there between), preferably between about 10 kb and 25 kb, more preferably between about 10-15 kb and 25 kb, and even more preferably between about 15 kb and 20 kb. Suitable plasmid vectors are commercially available. Ligation of the DNA into the plasmid is performed using methods well known in the art in view of the teachings of this specification.

In embodiments of the invention where it is desired to eliminate some of the sequences from the viral genomic nucleic acid of the construct, the amount of viral genomic nucleic acid present in the construct, excluding the heterologous sequences inserted, may be smaller. For example the total size of the viral genomic nucleic acid in the vector may be from 1 to 20 kb, preferably from 1 to 15 kb, more preferably from 3 to 12 kb, even more preferably from 5 to 10 kb in length, excluding the length of the heterologous coding sequences introduced.

In many embodiments of the invention the endogenous coding sequences naturally associated with the chosen endogenous gene expression regulatory unit will be deleted. This may be done by any suitable means, but in many embodiments will be done by PCR A two step PCR strategy may be used to delete the coding sequences. Unique restriction enzyme sites for a particular gene will be chosen; one inside the endogenous coding sequences, one outside the gene in the 5′ region, upstream and a third in the 3′ region, downstream of the gene. A PCR reaction is then carried out with primers that amplify from the 5′ unique restriction site to just upstream of the coding sequences. The downstream primer includes the sequence of the unique restriction site in the coding sequences. This gives a PCR product comprising the 5′ region of the gene, including the endogenous promoter and any other regulatory elements desired, but lacking the coding sequences, which includes the 5′ restriction site and the site internal to the coding sequences. The vector containing the wild type viral genomic nucleic acid is then digested with the restriction enzymes specific for the unique 5′ and internal sites and the 5′ region of the gene is excised, this is then replaced with the PCR product. Repeating the same set of steps for the 3′ end of the gene gives a resultant construct which has the original 5′ and 3′ ends of the gene, but in which the coding regions having been removed. All that remains of the coding sequences is the unique restriction site, into which the heterologous coding sequences can be inserted.

By generating constructs which have unique restriction sites where the endogenous coding sequences previously were, this means that any chosen heterologous coding sequences can then be inserted into the vector. In one embodiment the chosen heterologous coding sequences will be amplified by PCR using primers including the unique restriction site to allow easy cloning into the desired site. In some embodiments multiple unique sites maybe engineered downstream of a chosen promoter to allow maximum flexibility in cloning strategy.

Although, preferably, the constructs of the invention will be generated by starting from a single fragment of genomic viral nucleic acid and then modifying it, the same end result may be achieved using other strategies. For example regions of genomic nucleic acid may be assembled portion by portion. This may make it easier to introduce the necessary heterologous coding sequences. In some embodiments this may allow deletions to be effectively introduced into the genomic nucleic acid, such as the removal of unnecessary sequences from the viral genomic nucleic acid.

PCR may, in some embodiments, be used to obtain the single genomic nucleic acid fragment for subsequent modification or for the amplification of particular subregions of the genomic nucleic acid of the construct. PCR may also be used to introduce desired sequence modifications such as mutations and/or the introduction of a particular restriction site.

The constructs of the invention typically will not compirse a full viral genome, rather they will comprise one or more subregions of a viral genome. Typically, therefore, the constructs, on their own will lack the ability to give rise to an infectious viral particle. The construct may lack a viral origin of replication and/or one or more genes essential for the replication of the virus which the viral genomic nucleic acid of the construct is derived. The viral genomic nucleic acid sequences of the construct may lack packaging signals. The sequences may lack a particular gene encoding a protein included in the viral particle of the wild type virus or a protein involved in viral replication, the construct may contain no such genes. In some embodiments, the only sequences expressed from the viral nucleic acid sequences in the construct will be the heterologous coding sequences operably linked to th endogenous gen expression regulatory units.

In one embodiment of the invention the viral genomic nucleic acid sequences present in the construct may be shortened by the removal of some of the unnecessary sequences between the chosen endogenous gene expression regulatory units. Thus some, or all, of the intervening sequences from the end of the transcription terminator element associated with the heterologous coding sequences and the next endogenous gene expression regulatory unit operably lined to heterologous coding sequences may be deleted. In addition, or alternatively, some or all of the endogenous sequences between the 5′ and 3′ termini of the gene expression regulatory unit which do not form part of the unit itself may be deleted. This may make the constructs easier to manipulate and propagate. It may also mean that the chance of a recombination between wild-type viruses and the constructs of the invention is reduced.

In terms of the amount of extraneous sequences deleted, in total, in comparison to the size of the region in the viral genome corresponding to between the 5′ and 3′ termini of the viral genomic nucleic acid in the construct, there may be removal of more than 10%, preferably more than 20%, more preferably more than 30% and even more preferably more than 50% of the sequences. In some embodiments, up to 75%, preferably up to 85% and even more preferably up to 95% of the viral genomic nucleic acid sequences may be deleted. In some cases the length of endogenous sequences upstream of one or more of the endogenous gene expression regulatory units operably linked to a heterologous coding sequence may be less than 5 kb, preferably less than 2.5 kb, even more preferably less than 1 kb and still more preferably less than 500 bp. These may be the amount of endogenous sequences upstream of the endogenous promoter. The amount of endogenous sequences immediately downstream of the endogenous gen expression regulatory unit, and in particular downstream of the heterologous coding sequences may be of similar size.

Sequences may be removed from between all of the endogenous gene expression regulatory units or only between some of them. In some cases, all of the endogenous sequences, apart from those involved in the expression of the heterologous coding sequences, may be deleted. The deletion may be, for example, from at least 250 bp, preferably at least 1 kb, more preferably at least 2.5 kb and even more preferably at least 5 kb in size. The deletions introduced may correspond to single deletions between pairs of adjacent endogenous gene expression regulatory units or multiple deletions may also be introduced. The deletions may be restricted to non-coding sequences. Typically, the deletions will be introduced to reduce the size of the construct, rather than for the purposes of attenuation.

In some embodiments of the invention the endogenous gene expression regulatory until will consist of an endogenous promoter. In such embodiments some or all of the intervening sequences between the endogenous promoters operably linked to the heterologous coding sequences will be deleted. The region deleted may typically be any of the sizes specified herein. Other components of the endogenous gene such as transcribed non-coding sequences and/or enhancer elements may be excised any may be replaced with heterologous sequences.

The deletions may be introduced using any suitable technique. For example, a construct may be cut with restriction enzymes that digest on either side of the region to be deleted. The resulting vector may be purified from the undesired fragment and re-ligated to give a vector comprising the desired deletion. Other techniques such as PCR may be used to introduce the chosen deletions. Sequencing and restriction enzyme digests may be used to confirm that the intended deletions have been reduced. In order to select for constructs with the desired deletion during cloning, ligations maybe digested with a restriction enzyme that cuts inside the region to be deleted prior to transformation.

The removal of some, or all, extraneous sequences to reduce the size of vectors is also equally applicable to viral genomic nucleic acid containing constructs similar to the others discussed herein, which only differ in the respect that the endogenous promoters expressed at the same phase are operably linked to the coding sequences with which they are naturally associated, rather than heterologous coding sequences. For example, the viral genomic nucleic acid may be derived from HSV and the construct is intended to be used to generate an immune response against immediate early proteins such as ICP 0, 4, 22 and 27 which are expressed from the viral genomic nucleic acid under the control of their normal endogenous promoters. The removal of extraneous sequences from the sequences encoding the antigens to be expressed and the endogenous gene expression units that they are operably linked to is also equally beneficial for

this kind of genomic nucleic acid construct. Again, extraneous sequences between the termini of the gene expression regulatory unit may be removed to further decrease the size of the construct.

Accordingly, the invention also provides a method of generating a nucleic acid construct for direct administration to a subject to elicit an immune response in the subject, the method comprising:

(a) inserting viral genomic nucleic acid into a vector backbone, said viral genomic nucleic acid comprising at least two endogenous gene expression regulatory units which each comprise an endogenous promoter where the endogenous promoters of the units are active at the same phase in the viral cycle of the virus the viral genomic nucleic acid is derived from; and

(b) either prior to, at the same time, or after inserting the viral genomic nucleic acid into the vector backbone, deleting from the viral genomic nucleic acid some or all of the viral sequences, apart from the at least two endogenous gene expression regulatory units, which are present in the region of the viral genome corresponding to that between the 5′ and 3′ ends of the viral genomic nucleic acid of the construct

where the length of the viral genomic nucleic acid inserted into the vector backbone is from 1 to 50 kb.

The deletion introduced may be of a similar nature to any of those discussed herein and the constructs generated may have similar characteristics and utilities to any of the other constructs of the invention, apart from the fact that the endogenous gene expression regulatory units are linked to their natural coding sequences, rather than heterologous ones. Thus, coated particles, dosage receptacles, particle mediated delivery devices, etc may be generated using such constructs and the constructs may be used in methods of immunization and of obtaining gene expression as discussed elsewhere herein.

Administration of Constructs

The nucleic acid constructs and ancillary substances described herein may be administered by any suitable method. In a preferred embodiment, described below, the constructs are administered by coating a suitable construct (e.g., cosmids or plasmids) onto core carrier particles and then administering the coated particles to the subject or cells. However, the genomic fragments may also be delivered using other non-viral systems, e.g., naked nucleic acid delivery.

Although the constructs may be delivered by viral means, preferably they are not. Typically, therefore, the constructs will be delivered directly to the subject by non-viral means. Thus the construct may lack viral packaging signal sequences and/or a viral origin of replication. Typically, it will lack the viral packaging sequences and/or viral origin of replication native to the virus that the viral genomic nucleic acid is derived from. The constructs will preferably not require a helper virus and/or viral proteins provided in trans in order to replicate and in particular will not make use of a helper virus or proteins from the virus the genomic nucleic acid is derived from to replicate. In the case of cosmid based constructs, however, lambda proteins may be provided in trans and the virus may have the necessary cosmid sequences for replication.

In embodiments whre a separate nucleic acid construct is used to express an adjuvant, the construct may be formulated with a construct of the invention or separately. If formulated separately the method of formulation may the same employed to formulate the construct of the invention and/or the formulations my be the same as each other apart from the construct present. The two constructs may be administered at any suitably ratio, such as, for example, in equimolar amounts or in a 1:2, preferably 1:5, or more preferably 1:10 molar ratio with either construct being the one in excess. The invention also provides vaccines comprising a construct of the invention and a construct encoding adjuvant.

Conventional Pharmaceutical Preparations

Formulation of a preparation comprising a construct of the present invention, with or without addition of an adjuvant composition, can be carried out using standard pharmaceutical formulation chemistries and methodologies all of which are readily available to the ordinarily skilled artisan. For example, compositions containing one or more construct can be combined with one or more pharmaceutically acceptable excipients or vehicles to provide a liquid preparation.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like.

It is also preferred, although not required, that the preparation will contain a pharmaceutically acceptable excipient that serves as a stabilizer, particularly for peptide, protein or other like molecules if they are to be included in the vaccine composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane), see, e.g., Feigner et al. (1987) Proc. Natl. Acad. Sci. USA 84:7413-7416; Malone et al. (1989) Proc. Natl. Acad. Sci. USA 86:6077-6081; US Pat. Nos 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publication No. WO 93/19768).

Alternatively, the nucleic acid molecules of the present invention maybe encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated vaccine compositions will include a construct of the invention. An appropriate effective amount can be readily determined by one of skill in the art. Such an amount will fall in a relatively broad range that can be determined through routine trials. For example, immune responses have been obtained using as little as I pg of DNA, while in other administrations, up to 2mg of DNA has been used. It is generally expected that an effective dose of construct will fall within a range of about 10 μg to 1000 μg of construct, however, doses above and below this range may also be found effective. The compositions may thus contain from about 0.1% to about 99.9% of the construct.

Administration of Conventional Pharmaceutical Preparations

Administration of the above-described pharmaceutical preparations can be effected in one dose, continuously or intermittently throughout the course of treatment. Delivery will most typically be via conventional needle and syringe for the liquid compositions and for liquid suspensions containing particulate compositions. In addition, various liquid jet injectors are known in the art and may be employed to administer the present compositions. Methods of determining the most effective means and dosages of administration are well known to those of skill in the art and will vary with the delivery vehicle, the composition of the therapy, the target cells, and the subject being treated. Single and multiple administrations can be carried out with the dose level and pattern being selected by the attending physician.

Furthermore, it is also intended that the constructs delivered by the methods of the present invention be combined with other suitable compositions and therapies. For instance, in order to augment an immune response in a subject, the compositions and methods described herein can further include ancillary substances (e.g., adjuvants), such as pharmacological agents, cytokines, or the like. Ancillary substances maybe administered, for example, as proteins or other macromolecules at the same time, prior to, or subsequent to, administration of the DNA vaccines (e.g., cosmids or plasmids) described herein. The compositions may also be administered directly to the subject or, alternatively, delivered ex vivo, to cells derived from the subject, using methods known to those skilled in the art.

Coated Particles

In one embodiment, constructs of the invention, and other ancillary components such as adjuvants are delivered using carrier particles. Particle-mediated delivery methods for administering such nucleic acid preparations are known in the art. Thus, once prepared and suitably purified, the above-described constructs can be coated onto carrier particles (e.g., core carriers) using a variety of techniques known in the art. Carrier particles are selected from materials which have a suitable density in the range of particle sizes typically used for intracellular delivery from an appropriate particle delivery device. The optimum carrier particle size will, of course, depend upon the diameter of the target cells.

For the purposes of the present invention, core particles which may be used include tungsten, gold, platinum and iridium core carrier particles. Tungsten and gold particles are preferred. Tungsten particles are readily available in average sizes of 0.5 to 2.0 μm in diameter. Although such particles have optimal density for use in particle delivery methods, and allow highly efficient coating with nucleic acid, tungsten may potentially be toxic to certain cell types. Accordingly, gold particles or microcrystalline gold (e.g., gold powder A1570, available from Engelhard Corp., East Newark, N.J.) will also find use with the present methods. Gold particles provide uniformity in size (available from Alpha Chemicals in particle sizes of 1-3 μm, or available from Degussa, South Plainfield, N.J. in a range of particle sizes including 0.95 μm) and reduced toxicity.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in a suitable particle delivery device.

Peptide adjuvants (e.g., cytokines and bacterial toxins), can also be coated onto the same or similar core carrier particles. For example, peptides can be attached to a carrier particle by simply mixing the two components in an empirically determined ratio, by ammonium sulfate precipitation or other solvent precipitation methods familiar to those skilled in the art, or by chemical coupling of the peptide to the carrier particle. The coupling of L-cysteine residues to gold has been previously described (Brown et al., Chemical Society Reviews 2:271-311 (1980)). Other methods would include, for example, dissolving the peptide adjuvant in absolute ethanol, water, or an alcohol/water mixture, adding the solution to a quantity of carrier particles, and then drying the mixture under a stream of air or nitrogen gas while vortexing. Alternatively, the adjuvant can be dried onto carrier particles by centrifugation under vacuum. Once dried, the coated particles can be resuspended in a suitable solvent (e.g., ethyl acetate or acetone), and triturated (e.g., by sonication) to provide a substantially uniform suspension. The core carrier particles coated with the adjuvant can then be combined with core carrier particles carrying the nucleic acid constructs of the invention and administered in a single particle injection step, or administered separately from the nucleic acid construct compositions.

In some embodiments constructs encoding an adjuvant may be coated onto the same particles as the constructs of the invention or may be coated onto separate particles and then mixed with particles coated with a construct of the invention.

Administration of Coated Particles

Following their formation, core carrier particles coated with the constructs of the present invention, alone or in combination with e.g., adjuvant preparations, are delivered to a subject using particle-mediated delivery techniques.

Various particle delivery devices suitable for particle-mediated delivery techniques are known in the art, and are all suited for use in the practice of the invention. Current device designs employ an explosive, electric or gaseous discharge to propel the coated core carrier particles toward target cells. The coated particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 100.0 μg, more typically from 0.01 to 10.0 μg and preferably from 0.1 to 5 μg of nucleic acid molecule per dose, and in the case of peptide or protein molecules is 1 μg to 5 mg, more typically from 1 to 50 μg, preferably from 5 to 25 μg of peptide, depends on the subject to be treated.

In embodiments where a construct encoding an antigen is to be administered a similar amount of such a construct may be administered. Alternatively the total amount of the construct of the invention and the construct encoding the adjuvant may fall within the above ranges.

The exact amount of construct necessary will vary depending on the age and general condition of the individual being immunized and the particular nucleotide sequence or peptide selected, as well as other factors. An appropriate effective amount can be readily determined by one of skill in the art upon reading the present specification.

Thus, an effective amount of the constructs herein described will be sufficient to bring about a suitable immune response in an immunized subject, and will fall in a relatively broad range that can be determined through routine trials. Preferably, the coated core particles are delivered to suitable recipient cells in order to bring about an immune response (e.g., T-cell activation) in the treated subject.

Particulate Compositions

Alternatively, the constructs of the present invention, as well as one or more selected adjuvants, can be formulated as a particulate composition. More particularly, formulation of particles comprising a construct of interest can be carried out using standard pharmaceutical formulation chemistries and methodologies all of which are readily available to the person skilled in the art. For example, one or more construct and/or adjuvants can be combined with one or more pharmaceutically acceptable excipients or vehicles to provide a vaccine composition. In some embodiments a nucleic acid encoding an adjuvant, rather than the adjuvant itself, will be included in the composition. Auxiliary substances, such as wetting or emulsifying agents, pH buffering substances, and the like, may be present in the excipient or vehicle. These excipients, vehicles and auxiliary substances are generally pharmaceutical agents that do not themselves induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like.

It is also preferred, although not required, that the nucleic acid composition will contain a pharmaceutically acceptable carrier that serves as a stabilizer, particularly for peptide, protein or other like adjuvants or ancillary materials. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols LEGs), and combination thereof. A thorough discussion of pharmaceutically acceptable excipients, carriers, stabilizers and other auxiliary substances is available in REMINGTONS PHARMACEUTICAL SCIENCES (Mack Pub. Co., N.J. 1991), incorporated herein by reference.

The formulated compositions will be delivered in an amount sufficient to give rise to an immunological response, as defined above. An appropriate effective amount can be readily determined by one of skill in the art. Such an amount will fall in a relatively broad range, generally within the range of about 0.1 μg to 25 mg or more of the nucleic acid construct of interest, and specific suitable amounts can be determined through routine trials.

The compositions may contain from about 0.1% to about 99.9% preferably from 1 to 80%, more preferably from 10 to 50% and even more preferably from 20 to 40% of the nucleic acid molecule. If an adjuvant is included in the composition, or the methods are used to provide a particulate adjuvant composition, the adjuvant will be present in a suitable amount as described above. The compositions are then prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in commonly owned International Publication No. WO 97/48485, incorporated herein by reference.

Single unit dosages or multidose containers, in which the particles may be packaged prior to use, can comprise a hermetically sealed container enclosing a suitable amount of the particles comprising a suitable nucleic acid construct and/or the selected adjuvant (e.g., to provide a vaccine composition). The particulate compositions can be packaged as a sterile formulation, and the hermetically sealed container can thus be designed to preserve sterility of the formulation until use in the methods of the invention. If desired, the containers can be adapted for direct use in a particle delivery device. Such containers can take the form of capsules, foil pouches, sachets, cassettes, and the like. Appropriate particle delivery devices (e.g., needleless syringes) are described herein and may also be packaged with the particles for delivery.

The container in which the particles are packaged can further be labelled to identify the composition and provide relevant dosage information. In addition, the container can be labelled with a notice in the form prescribed by a governmental agency, for example the Food and Drug Administration, wherein the notice indicates approval by the agency under Federal law of the manufacture, use or sale of the antigen, adjuvant (or vaccine composition) contained therein for human administration.

The particulate compositions (comprising one or more construct of interest alone, or in combination with a selected adjuvant) can then be administered using a transdermal delivery technique. Preferably, the particulate compositions will be delivered via a powder injection method, e.g., delivered from a needleless syringe system such as those described in commonly owned International Publication Nos. WO 94/24263, WO 96/04947, WO 96/12513, and WO 96/20022, all of which are incorporated herein by reference. Delivery of particles from such needleless syringe systems is typically practised with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, more preferably about 1.2 to 1.4 g/cm³, and injection velocities generally range from about 100 to 3,000 m/sec, or greater. With appropriate gas pressure, particles having an average diameter of 10-70 μm can be accelerated through the nozzle at velocities approaching the supersonic speeds of a driving gas flow.

If desired, these needleless syringe systems can be provided in a preloaded condition containing a suitable dosage of the particles comprising the construct and/or the selected adjuvant. The loaded syringe can be packaged in a hermetically sealed container, which may further be labelled as described above.

Thus, the method can be used to obtain nucleic acid particles having a size ranging from about 10 to about 250 μm, preferably about 10 to about 150 μm, and most preferably about 20 to about 60 μm; and a particle density ranging from about 0.1 to about 25 g/cm³, and a bulk density of about 0.5 to about 3.0 g/cm³, or greater.

Similarly, particles of selected adjuvants having a size ranging from about 0.1 to about 250 μm, preferably from about 0.1 to about 150 μm, and most preferably from about 20 to about 60 μm; a particle density ranging from about 0.1 to about 25 g/cm³, and a bulk density of preferably about 0.5 to about 3.0 g/cm³, and most preferably about 0.8 to about 1.5 g/cm³ can be obtained.

Administration of Particulate Compositions

Following their formation, the particulate compositions (e.g., powder) can be delivered transdermally to the tissue of a vertebrate subject using a suitable transdermal delivery technique. Various particle delivery devices suitable for administering the substance of interest are known in the art, and will find use in the practice of the invention. A particularly preferred transdermal particle delivery system employs a needleless syringe to fire solid particles in controlled doses into and through intact skin and tissue. See, e.g., U.S. Pat. No. 5,630,796 to Belihouse et al. which describes a needleless syringe (also known as “the PowderJect® particle delivery device”). Other needleless syringe configurations are known in the art and are described herein.

Compositions containing a therapeutically effective amount of the powdered molecules described herein can be delivered to any suitable target tissue via the above-described particle delivery devices. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid constructs delivery is preferably to, and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 μg/kg to 100 μg/kg of nucleic acid construct per dose, depends on the subject to be treated. Doses for other pharmaceuticals, such as physiological active peptides and proteins, generally range from about 0.1 μg to about 20 mg, preferably 10 μg to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

Thus, a “therapeutically effective amount” of the present particulate compositions will be sufficient to bring about treatment or prevention of disease or condition symptoms, and will fall in a relatively broad range that can be determined through routine trials.

Below are examples of specific embodiments for carrying out the present invention. The Examples are offered for illustrative purposes only, and are not intended to limit the scope of the present invention in any way.

### EXAMPLES

Efforts have been made to ensure accuracy with respect to numbers used (e.g., amounts, temperatures, etc.), but some experimental error and deviation should, of course, be allowed for.

### Example 1

### Construction of the OP23-6 Construct

A HSV-2 construct comprising all four immediate early genes, but lacking extraneous viral genomic sequences was constructed. The starting point for constructing the vector was a cosmid that included three EcoRI fragments from the HSV-2 MS strain spanning nucleotides 110,931 to 147,530 of the HSV-2 genome based on the published sequence (HG52 strain). The gene order is also as shown in the published sequence.

The cosmid was partially digested with EcoRI and religated and a construct that had only the 28,000 fragment (110,931-139,697) was selected. This molecule was designated OP-23. From this molecule six modifications were made to remove the majority of the unnecessary sequences from the viral genomic nucleic acid. The modifications were as follows:
1. Bst1107I and ScaI digestion and religation of the cosmid (removes the ampicillin resistance gene) to create OP23-1.
2. NsiI digestion and religation to remove the SV40 origin of replication and create OP23-2.
3. BstXI partial digestion and religation to remove regions between ICP27 and ICP0 to give OP23-3.
4. Complete digest with BspHI, followed by partial digestion with BsiWI and then religation to remove sequences following the ICP22 gene and some backbone sequences. This gives OP23-4.
5. Srft digest and religation to create OP23-5 (removes sequences between ICP4 and ICP0).
6. BstXI total digestion and religation to create OP23-6 (removes a small fragment from between ICP27 and ICP0).

Sequencing of the OP23-6 construct was carried out to confirm the structure of the vector and its sequence.

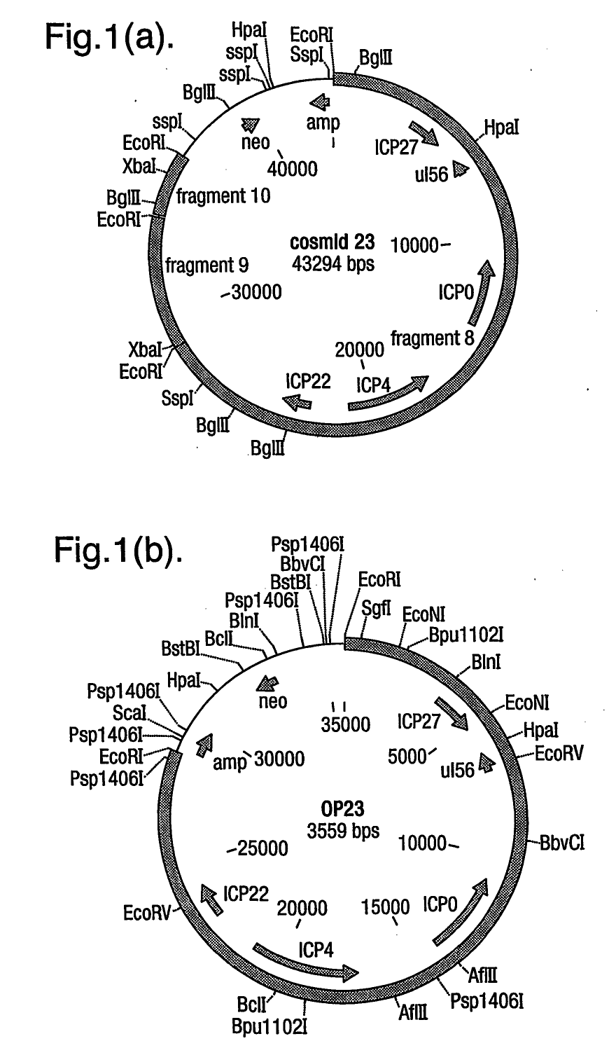

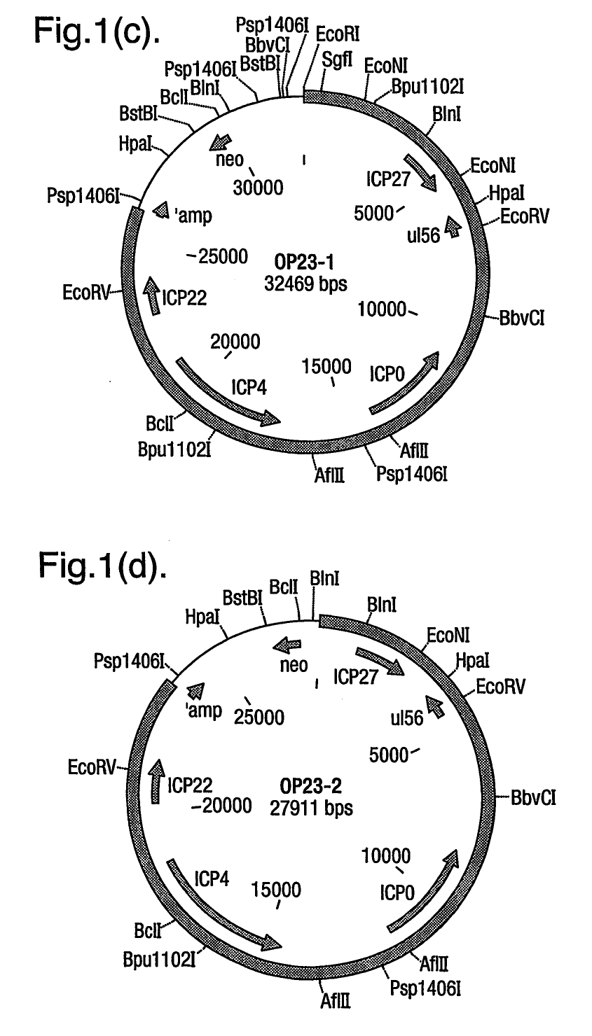

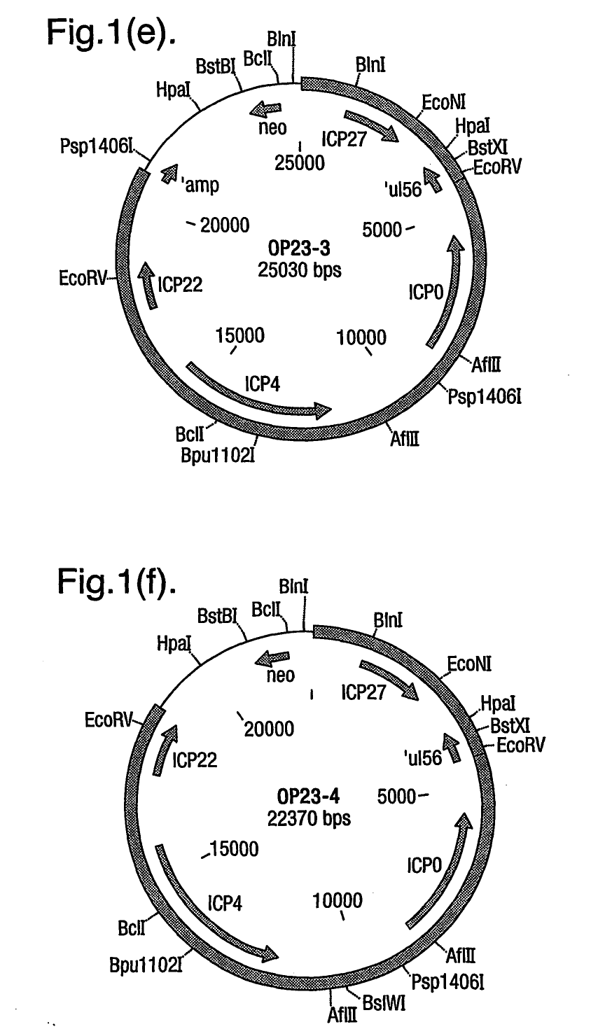

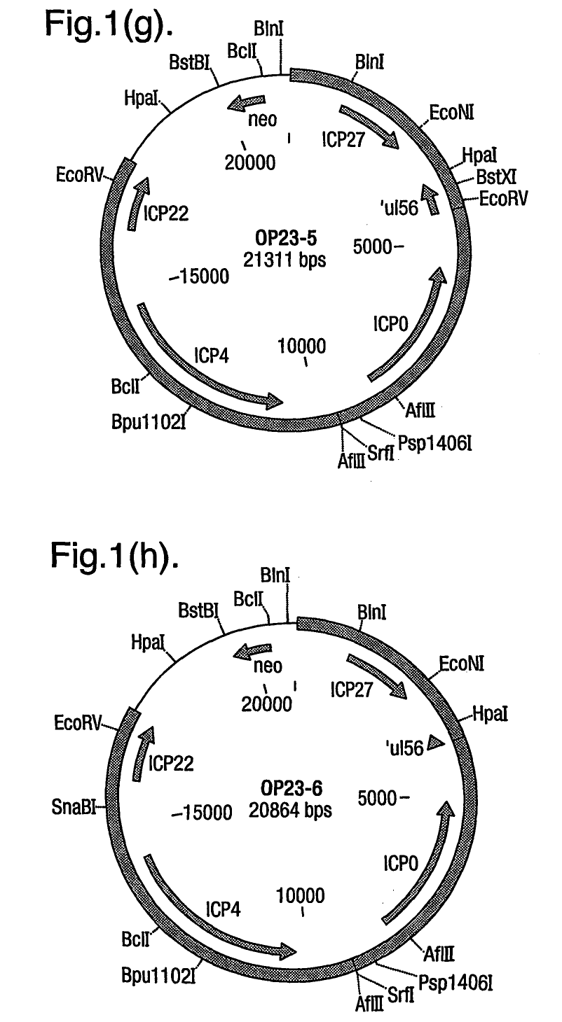

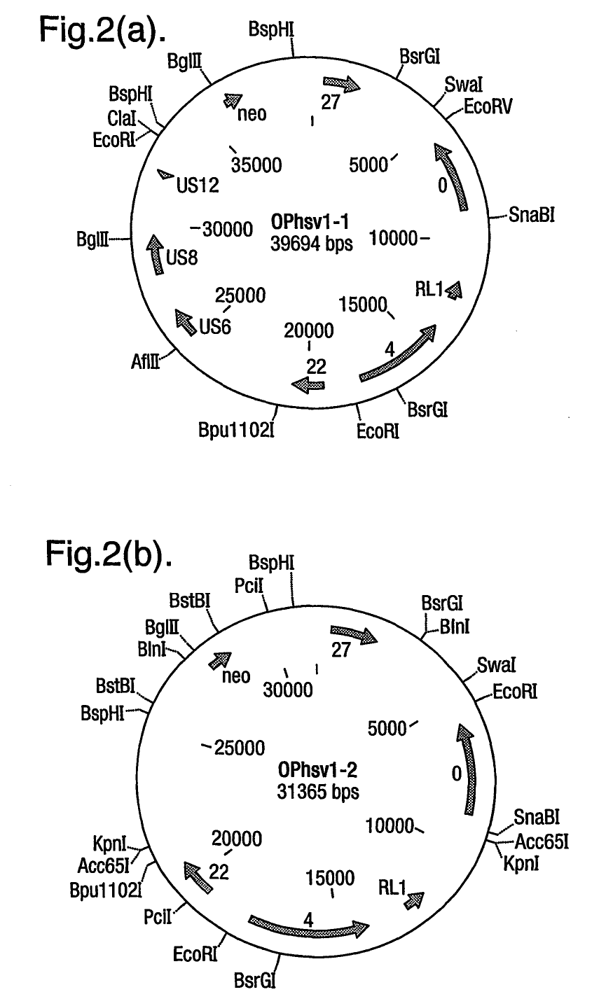

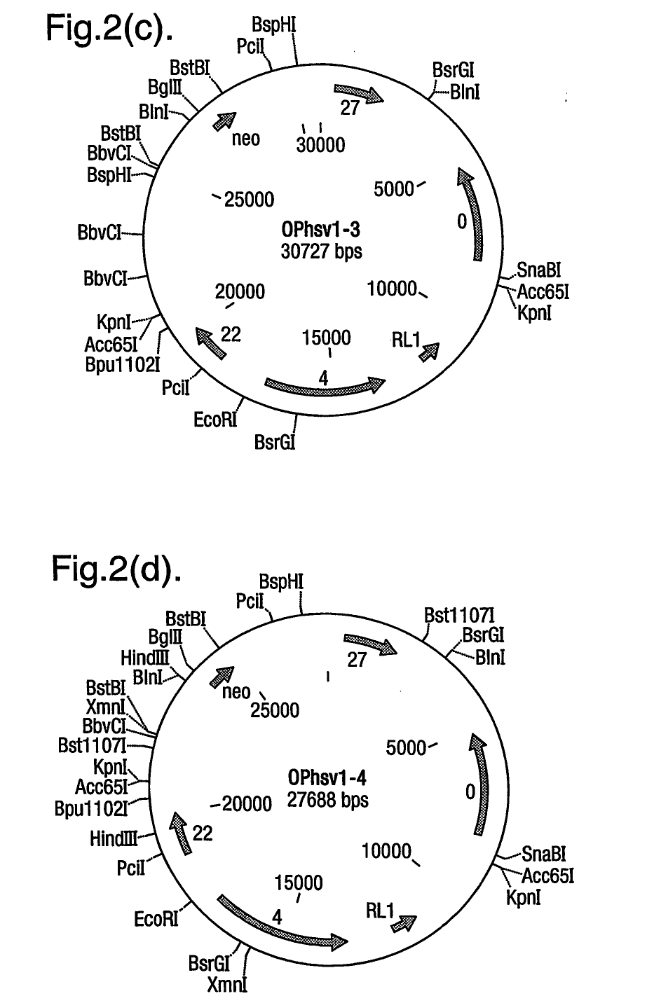

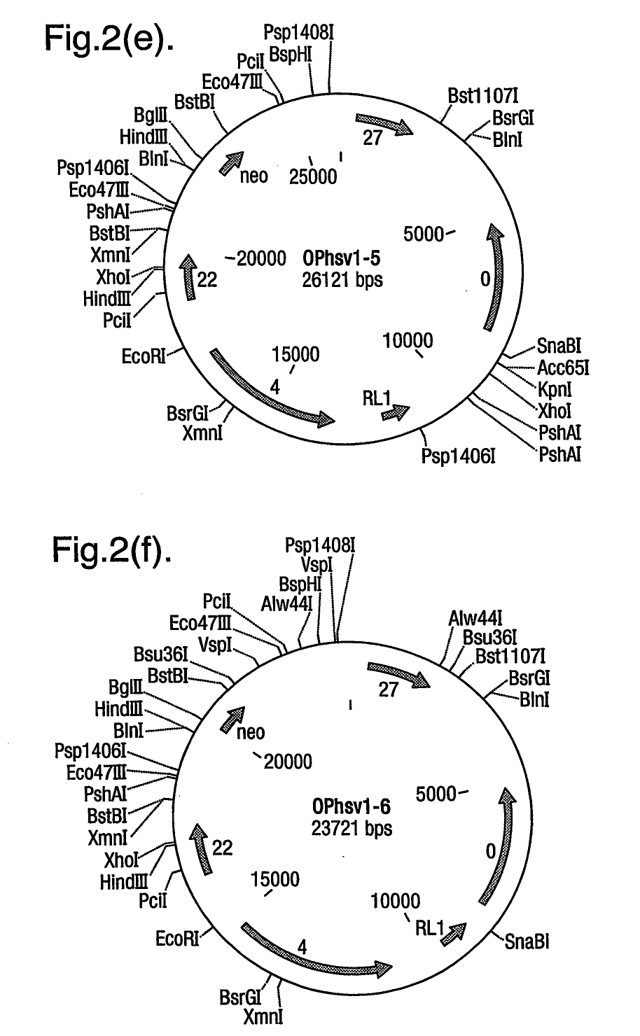

The structure of constructs OP23 and OP23-1 to OP23-6 is shown in FIG. 1.

### Example 2

### Generation of Multi-Antigen HSV1 Vaccine Lacking Extraneous Sequences

A similar type of vaccine for HSV-1 can be developed to that described in Example 1 for HSV-2.

An EcoRI partial digest would be performed on HSV-1 to generate genomic fragments, cosmids comprising the fragments would then be generated using the superCos kit from Stratagene. A cosmid is then selected which contains the sequences from 110,095 to 146,694 of the HSV-1 genome. Several digests and religations can then be performed to produce a final compact vector expressing the four immediate early genes from HSV-1, but lacking the majority of the unnecessary intervening sequences:

The following steps are carried out to generate the desired construct:
1. Cosmid HSV1 (43392 bp) is digested with ScaI and NdeI and religated to give OPhsv1-1.
2. OPhsv1-1 (39694 bp) is digested with AflII and ClaI and religated to give OPhsv1-2.
3. OPhsv1-2 (31365 bp) is digested with EcoRV and Swa I and religated to give OPhsv1-3.
4. OPhsv1-3 (30727 bp) is digested with BbvCI and religated to give to give OPhsv1-4.
5. OPhsv1-4 (27688 bp) is digested with BpuI 1021 and BbvC1 and religated to give OPhsv1-5.
6. OPhsv1-5 (26121 bp) is digested with kpn and partially digested with Psp14061 and, religated to give OPhsv1-6 the final construct which has all four immediate early genes present, but from which has the majority of other extraneous sequences have been removed.

### Example 3

### Insertion of Heterologous Coding

(i) General Strategy

The OP23-6 construct generated in Example 1 is used to generate a construct where the ICP 0, 4, 22 and 27 coding sequences are replaced by heterologous coding sequences. The basic strategy employed is to first remove the original coding sequences and then to insert the heterologous coding sequences in their place. For each coding sequence to be replaced the concept is to find three unique restriction enzyme sites; one inside the gene; one outside the gene in the 5′ region, upstream; and one in the 3′ region, downstream. The coding sequence is then replaced in two steps.

PCR primers are chosen to amplify from, and including, the 5′ upstream unique restriction site to just upstream of the start of the coding sequences. The 3′ primer includes the sequence of the unique restriction site in the coding sequences. The PCR produces a DNA fragment that has the 5′ region of the gene, no coding sequences and is tailed with the unique restriction site found in the coding sequences. The original vector is then digested with the unique enzyme specific for the 5′ restriction site and the restriction site internal to the coding sequence to excise the 5′ half of the gene. This is then replaced with the PCR product digested with the same enzymes. Repeating the same set of steps for the 3′ end of the coding sequences gives a resultant construct that has the original 5′ and 3′ ends of the gene, but in which the coding regions have been removed. All that remains of the construct is the unique enzyme site inside it. The new gene will then be inserted into the site. The process can then be repeated for as many of the ICP genes present in OP23-6 as desired.

(ii) Molecular Biology

Standard molecular biology methodology used for manipulating DNA sequences is utilized for the conversion of OP23-6 into the desired multi-genic construct, expressing heterologous antigens. To generate appropriate DNA fragments to replace segments of the original OP23-6, Polymerase Chain Reaction (PCR) is carried out and the fragments are cloned into the pTARGET vector (Promega). Positive clones are then identified by restriction digest and DNA purified from bacterial culture of the positive clones is used to isolate pure preparations of the desired fragments. Fragments purified from agarose gels are ligated into the OP23-6 vector which has previously been cut with the appropriate restriction enzymes and purified from agarose gels. Positives are then screened for by restriction digests.

The heterologous genes to be inserted into the vector are obtained by PCR reactions in which the appropriate restriction sites were engineered into the 5′ and 3′ ends of the PCR fragments. Positives containing the desired insert are screened for by restriction digest and the proper orientation is confirmed as well. DNA sequencing is carried out to confirm that the resulting clones contain the desired sequences.

(iii) Preparation of DNA Vaccines.

Precipitation of DNA onto gold particles is achieved using standard procedures for the calcium/spermidine formulation of DNA vaccines. DNA is mixed with 2 micron gold particles in a small centrifuge tube containing 300 ml of 50 mM spermidine. The amount of DNA added is 2 μg per mg gold particles and typically batches of 26 mg gold (52 μg of DNA) are made. The DNA is precipitated onto gold by the addition of a 1/10 volume of 10% CaCl₂ during continuous agitation of the tube on a rotary mixer. DNA-gold complexes are washed three times with absolute ethanol and then loaded into Tefzel tubing, dried and cut into 0.5 inch segments for use in the XR-1 device.

For immunization, DNA vaccines are delivered by the XR-1 device into the abdomen of Balb/C mice. A single shot is given for each immunization and animals are given a prime and a boost at 4 weeks. Samples are collected from animals two weeks after the final immunization.

(iv) Antibody ELISA Serum samples are assayed for antibodies against the heterologous antigens expressed by the vector using an ELISA assay. Falcon Pro Bind microtiter plates are coated overnight at 4° C. with antigen in PBS (phosphate buffered saline, BioWhittaker). The plates are blocked for 1 hour at room temperature with 5% dry milk/PBS, then washed three times with wash buffer (10 mM Tris Buffered saline, 0.1% Brij-35). Serum samples diluted in dilution buffer (2% dry milk/PBS/0.05% Tween 20) are added to the plate and then incubated for 2 hours at room temperature. Plates are washed three times and a biotinylated goat anti-mouse antibody (Southern Biotechnology) diluted 1:8000 in dilution buffer is added to the plate and incubated for 1 hr at room temperature. Following the incubation, plates are washed three times, then a Streptavidin-Horseradish peroxidase conjugate (Southern Biotechnology) diluted 1:8000 in PBS is added and the plate incubated a further 1 hr at room temperature. Plates are washed three times, then substrate solution is added (BioRad) and the reaction is stopped with 1N H₂SO₄. Optical density is read at 450 nm.

(v) Cell Culture

Single cell suspensions are obtained from mouse spleens. Spleens are squeezed through a mesh to produce a single cell suspension and cells are then sedimented, and treated with ACK buffer (Rio Whittaker, Walkersville Md.) to lyse red blood cells. The cells are then washed twice in RPMI 1640 media supplemented with HEPES, 1% glutamine (Bio Whittaker), and 5% heat inactivated fetal calf serum (FCS, Harlan, Indianapolis Ind.). Cells are counted, and resuspended to an appropriate concentration in “Total” media consisting of RPMI 1640 with HEPES and 1% glutamine, supplemented with 5% heat inactivated FCS, 50 mM mercaptoethanol (Gibco-BRL, Long Island N.Y.), gentamycin (Gibco-BRL), 1 mM MEM sodium pyruvate (Gibco-BRL) and MEM non-essential amino acids (Sigma, St. Louis Mo.). Cell suspensions are then utilized in various immunoassays. For CD8 specific assays cells are cultured in vitro in the presence of a peptide corresponding to a known CD8 epitopes. Peptides are made up in DMSO (10 mg/ml) and diluted to 10 ug/ml in culture medium.

(v) ELISPOTs

For IFN-g ELISPOTs assays Millipore Multiscreen membrane filtration plates are coated with 50 μl of 15 μg/ml anti-IFN-g antiserum (Pharmingen) in sterile 0.1M carbonate buffer pH 9.6, overnight at 4° C. Plates are washed six times with sterile PBS and then blocked with tissue culture medium containing 10% fetal bovine serum (FBS) for 1-2 hr at room temperature. The medium is removed and spleen cells dispensed into the wells with a total of 1×10⁶ cells per well. For wells in which less than 1×10⁶ cells from immunized animals are added, cells from naive animals are used to bring the total to 1×10⁶. Cells are incubated overnight in a tissue culture incubator in the presence of the peptide as described above. Plates are washed two times with PBS and one time with distilled water. This is followed with three washes with PBS. Biotinylated anti IFN-g monoclonal antibody (Pharmingen) is added to the plate (50 ul of a 1 ug/ml solution in PBS) and incubated for 2 hr at room temperature. Plates were washed six times with PBS then 50 μl of a Streptavidin Alkaline phosphatase conjugate (1:1000 in PBS, Pharmingen) is added and incubated for 2 hr at room temperature. Plates are washed six times with PBS and the color substrate (BioRad) is added and the reaction is allowed to proceed until dark spots appear. The reaction is stopped by washing with water three times. Plates are air-dried and spots counted under a microscope.

1. A nucleic acid construct comprising viral genomic nucleic acid, said viral genomic nucleic acid comprising at least two endogenousgene expression regulatory units which each comprise an endogenous promoter capable of expression in a mammalian cell, where the endogenous promoters of the units are active at the same phase in the viral life cycle of the virus the viral genomic nucleic acid is derived from, where:
(a) at least two of the endogenous gene expression regulatory units comprising promoters active at the same phase are each operably linked to a separate heterologous coding sequence inserted into the viral genomic nucleic acid; and
(b) the viral genomic nucleic acid is from 1 to 50 kb in length excluding the heterologous sequences inserted into it

2. A nucleic acid construct according to claim 1, wherein the at least two endogenous promoters are switched on at the same point in the viral life cycle of the virus the genomic nucleic acid is derived from

3. A nucleic acid construct according to claim 1, wherein the at least two endogenous gene expression regulatory units are either both/all from immediate early or both/all from early viral genes.

4. A nucleic acid construct according to claim 1, wherein the at least two endogenous gene expression regulatory units are different

5. A nucleic acid construct according to claim 1, wherein the virus the viral genomic nucleic acid is derived from is selected from the group consisting of a DNA virus and an RNA virus.

6. A nucleic acid construct according to claim 5, wherein the DNA virus is a double stranded DNA virus selected from a herpesvirus and an adeno associated virus (AAV).

7. A nucleic acid construct according to claim 6, wherein the herpesvirus is selected from the group consisting of a herpes simplex virus (HSV), a cytomegalovirus (CMV) and an Epstein Barr virus (EBV).

8. A nucleic acid construct according to claim 7, wherein the HSV is selected from the group consisting of HSV-1 and HSV-2.

9. A nucleic acid construct according to claim 7, wherein the viral genomic nucleic acid is derived from a herpes sinmplex virus and the at least two endogenous gene expression regulatory units each comprise an endogenous promoter selected from the group consisting of the ICP0, ICP4, ICP22 and ICP27 gene promoters.

10. A nucleic acid construct according to claim 7, wherein the viral genomic nucleic acid is derived from a herpes simplex virus and the two endogenous promoters of the at least two gene expression regulatory units are HSV tegument protein gene promoters.

11. A nucleic acid construct according to claim 7, wherein the viral genomic nucleic acid is from human cytomegalovirus and the endogenous promoters of the at least two gene expression regulatory units are:
at least two selected from the group consisting of the UL36, UL37 and UL38 gene promoters;
the UL82 and UL83 gene promoters; or
the UL122 and U123 gene promoters.

12. A nucleic acid construct according to claim 1, wherein all of the heterologous coding sequences expressed by the endogenous gene expression regulatory units are derived from the same organism.

13. A nucleic acid construct according to claim 1, wherein two or more of the heterologous coding sequences encode antigens.

14. A nucleic acid construct according to claim 1, wherein the antigens are antigens from a pathogen.

15. A nucleic acid construct according to claim 1, wherein some or all of the viral sequences, apart from the at least two endogenous gene expression regulatory units, which are present in the region of the viral genome corresponding to that between the 5′ and 3′ ends of the viral genomic nucleic acid in the construct are absent from the construct

16. A nucleic acid construct according to claim 15, wherein the absent region comprises part or all of the intervening sequences between two of the adjacent endogenous gene expression regulatory units linked to heterologous coding sequences.

17. A nucleic acid construct according to claim 15, wherein the absent region corresponds to one or more of the genes present in the region of the viral genome other than those of the at least two endogenous gene expression regulatory units used to express the heterologous coding sequences.

18. A nucleic acid construct according to claim 15, wherein the viral genomic nucleic acid is from HSV-2 and the viral sequences have been removed from the construct by one or more of the following techniques:
(a) a partial digestion with a BstXI enzyme and then religation to remove sequences between ICP27 and ICP0;
(b) a complete digestion with a BspHI enzyme, followed by a partial digestion with a BsiWI enzyme and then religation to remove sequences adjacent to ICP22;
(c) a digestion with a SrfI enzyme and then religation to remove sequences between ICP4 and ICP0; and
(d) total digestion with a Bst[ enzyme and then religation to remove sequences between ICP27 and ICP0.

19. A nucleic acid construct according to claim 15, wherein the viral genomic nucleic acid is from HSV-I and the viral sequences have been removed from the construct to remove substantially all of the HSV-1 sequences extraneous to ICP0, ICP4, ICP22 and ICP27 coding sequences.

20. A nucleic acid construct according to claim 1, wherein the viral genomic nucleic acid corresponds to a contiguous region of the viral genome it is derived from apart from the replacement of the coding sequences the endogenous gene expression regulatory units are naturally operably linked to with the heterologous coding sequences.

21. A nucleic acid construct according to claim 1, wherein the endogenous gene expression regulatory units operably linked to the heterologous coding sequences are endogenous promoters.

22. A method of generating a nucleic acid construct for direct administration to a subject to elicit an immune response in the subject, the method comprising:
(a) inserting viral genomic nucleic acid into a vector backbone, said viral genomic nucleic acid comprising at least two endogenous gene expression regulatory units which each comprise an endogenous promoter capable of expression in a mammalian cell, where the endogenous promoters of the units are active at the same phase in the viral cycle of the virus the viral genomic nucleic acid is derived from; and
(b) either prior to, at the same time, or subsequent to inserting -the viral genomic nucleic acid into the vector backbone, operably linking each of the endogenous promoters of at least two of the gene expression regulatory units in the viral genomic nucleic acid to heterologous coding sequences
wherein the viral genomic nucleic acid is from 1 to 50 kb in length excluding the heterologous sequences inserted into it.

23. A method according to claim 22, wherein the method further comprises deleting from the viral genomic nucleic acid some or all of the viral sequences, apart from the at least two endogenous gene expression regulatory units, which are present in the region of the viral genome corresponding to that between the 5′ and 3′ ends of the viral genomic nucleic acid of the construct

24. A method according to claim 23, wherein the deleted sequences are some or all of the non-coding intervening sequences between adjacent endogenous gene expression regulatory units to which the heterologous coding sequences are to be operably linked

25. A method according to claim 22, wherein the genomic nucleic acid is inserted into the vector backbone as a single fragment.

26. Coated particles, suitable for delivery from a particle-mediated delivery device, which particles comprise carrier particles coated with a nucleic acid construct wherein the construct comprises viral genomic nucleic acid, said viral genomic nucleic acid comprising at least two endogenous gene expression regulatory units which each comprise an endogenous promoter capable of expression in a mammalian cell, where the endogenous promoters of the units are active at the same point in the viral cycle of the virus the viral genomic nucleic acid is derived from, where:
at least two of the endogenous gene expression regulatory units comprising gene expression regulatory units comprising promoters are each operably linked to a heterologous coding sequence inserted into the-viral genomic nucleic acid; and
the viral genomic nucleic acid is from 1 to 50 kb in length excluding the heterologous sequences inserted into it.

27. Coated particles according to claim 26, wherein the carrier particles are gold or tungsten.

28. A dosage receptacle for a particle mediated delivery device comprising coated particles according to claim 26.

29. A particle mediated delivery device loaded with coated particles according to claim 26.

30. A particle mediated delivery device according to claim 29 which is a needleless syringe.

31. A method of obtaining expression in a mammalian cell of a polypeptide of interest, which method comprises transferring into said cells a nucleic acid construct comprising viral genomic nucleic acid, said viral genomic nucleic acid comprising at least two endogenous gene expression regulatory units which each comprise an endogenous promoter capable of expression in a mammalian cell, where the endogenous promoters of the units are active at the same phase in the viral cycle of the virus the viral genomic nucleic acid is derived from, where:
at least two of the endogenous gene expression regulatory units comprising promoters are each operably linked to a heterologous coding sequence inserted into the viral genomic nucleic acid; and
the viral genomic nucleic acid is from 1 to 50 kb in length excluding the heterologous sequences inserted into it.

32. A method according to claim 31, wherein the construct is delivered directly into a subject.

33. A method according to claim 32, wherein the construct is delivered by injection, transdermal particle delivery, inhalation, topically, orally, intranasally or transmucosally.

34. A method according to claim 32, wherein the construct is delivered by needleless injection.

35. A method according to claim 34, wherein the nucleic acid construct is coated onto carrier particles.

36. A method of nucleic acid immunisation comprising administering to a subject an effective amount of coated particles, which particles are suitable for delivery from a particle-mediated delivery device, the particles comprising carrier particles coated with a nucleic acid construct, wherein the construct comprises viral genomic nucleic acid, said viral genomic nucleic acid comprising at least two endogenous gene expression regulatory units which each comprise an endogenous promoter capable of expression in a mammalian cell, where the endogenous promoters of the units are active at the same phase in the viral cycle of the virus the viral genomic nucleic acid is derived from, where:
at least two of the endogenous gene expression regulatory units comprising promoters are each operably linked to a heterologous coding sequence inserted into the viral genomic nucleic acid; and
the viral genomic nucleic acid is from 1 to 50 kb in length excluding the heterologous sequences inserted into it

37. A method of generating a nucleic acid construct for direct administration to a subject to elicit an immune response in the subject, the method comprising:
(a) inserting viral genomic nucleic acid into a vector backbone, said viral genomic nucleic acid comprising at least two endogenousgene expression regulatory units which each comprise an endogenous promoter capable of expression in a mammalian cell, where the endogenous promoters of the units are active at the same phase in the viral cycle of the virus the viral genomic nucleic acid is derived from; and
(b) either prior to, at the same time, or after inserting the viral genomic nucleic acid into the vector backbone, deleting from the viral genomic nucleic acid some or all of the viral sequences, apart from the at least two endogenous gene expression regulatory units, which are present in the region of the viral genome corresponding to that between the 5′ and 3′ ends of the viral genomic nucleic acid of the construct
where the length of the viral genomic nucleic acid inserted into the vector backbone being from 1 to 50 kb.

38. A method according to claim 37, wherein the nucleic acid sequences deleted are part or all of the non-coding intervening sequences between two of the endogenous promoters.

39. Coated particles, suitable for delivery from a particle-mediated delivery device, which particles comprise carrier particles coated with a nucleic acid construct generated by a method as defined in claim 36.

40. A dosage receptacle for a particle mediated delivery device comprising coated particles according to claim 39.

41. A particle mediated delivery device loaded with coated particles according to claim 40.

42. A method of obtaining expression in a mammalian cell of a polypeptide of interest, which method comprises transferring into said cells a nucleic acid construct generated by a method according to claim 37.

43. A method of nucleic acid immunisation comprising administering to a subject an effective amount of coated particles, which particles are suitable for delivery from a particle-mediated delivery device, the particles comprising carrier particles coated with a nucleic acid construct generated by a method according to claim 37.

44. Use of a nucleic acid construct according to any one of claims 1 to 21, a nucleic acid construct generated by a method according to any one of claims 22 to 25, 37 and 38 or coated particles according to any one of claims 26, 27, and 39 in the manufacture of a medicament for use in nucleic acid immunisation.
